(19) United States
(12) Patent Application Publication
(10) Pub. No.: US 2008/0200512 A1
(43) Pub. Date: Aug. 21, 2008

(54) Hepatitis C virus polymerase inhibitors

(76) Inventors: Jeffrey Mark Blaney, Piedmont, CA (US); Normand Hebert, Encinitas, CA (US); Stephanie Ann Hopkins, Poway, CA (US); Elizabeth A. Jefferson, La Jolla, CA (US); Masaki Tomimoto, Osaka (JP)

Correspondence Address: ROCHE PALO ALTO LLC;PATENT LAW DEPT. M/S A2-250, 3431 HILLVIEW AVENUE, PALO ALTO, CA, 94304, US

(21) Appl. No.: 12/070,027

(22) Filed: Feb. 14, 2008

Related U.S. Application Data
(60) Provisional application No. 60/901,442, filed on Feb. 15, 2007.

(51) Int. Cl. A61K 31/445; C07D 211/60; A61P 31/12

(52) U.S. Cl. 514/330; 546/226

(57) ABSTRACT

The invention provides a compound of formula:

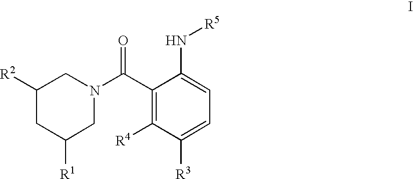

and methods for making and using the same, where R¹ and R² are independently hydrogen, C₁₋₆ alkyl or C₁₋₆ haloalkyl; R³ is hydrogen, halogen, C₁₋₆ alkyl or C₁₋₆ alkoxy; R⁴ is hydrogen or halogen; and R⁵ is a mono- or di-amino acid moiety, a succinic acid moiety, a urea moiety, or a derivative thereof. The invention also provides pharmaceutical compositions comprising a compound of formula I, and methods for making and using the same.

### CROSS REFERENCE TO PROP APPLICATIONS

This application claims the benefit of priority to U.S. Ser. No. 60/901,442 filed Feb. 15, 2007 which is hereby incorporated by reference in its entirety.

### FIELD OF THE INVENTION

The invention relates to inhibitors of hepatitis C virus (HCV) replicon RNA replication. In particular, the invention is concerned with the use of non-nucleoside heterocyclic compounds as inhibitors of subgenomic HCV RNA replication and pharmaceutical compositions containing such compounds.

Hepatitis C virus is the leading cause of chronic liver disease throughout the world. Boyer, N. et al. J. Hepatol., 2000, 32, 98-112. Patients infected with HCV are at risk of developing cirrhosis of the liver and subsequent hepatocellular carcinoma, and hence HCV is one of the major indications for liver transplantation.

HCV has been classified as a member of the virus family Flaviviridae that includes the genera flaviviruses, pestiviruses, and hapaceiviruses, which includes hepatitis C viruses. Rice, C. M., Flaviviridae: The viruses and their replication. In: Fields Virology, Editors: B. N. Fields, D. M. Knipe and P. M. Howley, Lippincott-Raven Publishers, Philadelphia, Pa., 1996, Chapter 30, 931-959. HCV is an enveloped virus containing a positive-sense single-stranded RNA genome of approximately 9.4 kb. The viral genome consists of a 5′-untranslated region (UTR), a long open reading frame encoding a polyprotein precursor of approximately 3011 amino acids, and a short 3′-UTR. The 5′-UTR is the most highly conserved part of the HCV genome and is important for the initiation and control of polyprotein translation.

Genetic analysis of HCV has identified six main genotypes which diverge by over 30% of the DNA sequence. More than 30 subtypes have been distinguished. In the US approximately 70% of infected individuals have Type 1a and 1b infection. Type 1b is the most prevalent subtype in Asia. See, for example, X. Forns and J. Bukh, Clinics in Liver Disease 1999, 3, 693-716; J. Bukh et al., Semin. Liv. Dis., 1995, 15, 41-63. Unfortunately Type 1 infection is more resistant to therapy than either type 2 or 3 genotypes (N. N. Zein, Clin. Microbiol. Rev., 2000, 13, 223-235).

Viral structural proteins include a nucleocapsid core protein (C) and two envelope glycoproteins, E1 and E2. HCV also encodes two proteases, a zinc-dependent metalloproteinase encoded by the NS2-NS3 region and a serine protease encoded in the NS3 region. These proteases are required for cleavage of specific regions of the precursor polyprotein to produce mature peptides. The carboxyl half of nonstructural protein 5, NS5B, contains the RNA-dependent RNA polymerase. The function of the remaining nonstructural proteins, NS4A and NS4B, and that of NS5A (the amino-terminal half of nonstructural protein 5) remain unknown. It is believed that most of the non-structural proteins encoded by the HCV RNA genome are involved in RNA replication

Currently there are only a limited number of approved therapies available for the treatment of HCV infection. New and existing therapeutic approaches to treating HCV and inhibition of HCV NS5B polymerase have been reviewed. See, for example, R. G. Gish, Sem. Liver. Dis., 1999, 19, 5; Di Besceglie, A. M. and Bacon, B. R., Scientific American, October: 1999, 80-85; G. Lake-Bakaar, Current and Future Therapy for Chronic Hepatitis C Virus Liver Disease, Curr. Drug Targ. Infect Dis., 2003, 3(3), 247-253; P. Hoffmann et al., Recent patents on experimental therapy for hepatitis C virus infection (1999-2002), Exp. Opin. Ther. Patents 2003, 13(11), 1707-1723; M. P. Walker et al., Promising Candidates for the treatment of chronic hepatitis C, Exp. Opin. investing. Drugs, 2003, 12(8), 1269-1280; S.-L. Tan et al., Hepatitis C Therapeutics: Current Status and Emerging Strategies, Nature Rev. Drug Discov., 2002, 1, 867-881; J. Z. Wu and Z. Hong, Targeting NS5B RNA-Dependent RNA Polymerase for Anti-HCV Chemotherapy, Curr. Drug Targ.—Infect. Dis., 2003, 3(3), 207-219.

Ribavirin (1-((2R,3R,4S,5R)-3,4-dihydroxy-5-hydroxymethyl-tetrahydro-furan-2-yl)-1H-[1,2,4]triazole-3-carboxylic acid amide; Virazole®) is a synthetic, non-interferon-inducing, broad spectrum antiviral nucleoside analog. Ribavirin has in vitro activity against several DNA and RNA viruses including Flaviviridae (Gary L. Davis. Gastroenterology, 2000, 118, S104-S114). Although, in monotherapy ribavirin reduces serum amino transferase levels to normal in 40% or patients, it does not appear to lower serum levels of HCV-RNA. Ribavirin also exhibits significant toxicity and is known to induce anemia. Viramidine is a prodrug that is converted to ribavirin in hepatocytes.

Interferons (IFNs) have been available for the treatment of chronic hepatitis for nearly a decade. IFNs are glycoproteins produced by immune cells in response to viral infection. Two distinct types of interferon are recognized: Type 1 includes several interferon alphas and one interferon β, type 2 includes interferon γ. Type 1 interferons are produced mainly by infected cells and protect neighboring cells from de novo infection. IFNs inhibit viral replication of many viruses, including HCV, and when used as the sole treatment for hepatitis C infection, IFN suppresses serum HCV-RNA to undetectable levels. Additionally, IFN normalizes serum amino transferase levels. Unfortunately, the effects of IFN are temporary. Cessation of therapy results in a 70% relapse rate and only 10-15% exhibit a sustained virological response with normal serum alanine transferase levels. (Davis, Luke-Bakaar, supra)

One limitation of early IFN therapy was rapid clearance of the protein from the blood. Chemical derivatization of IFN with polyethyleneglycol (PEG) has resulted in proteins with substantially improved pharmacokinetic properties. PEGASYS® is a conjugate interferon α-2a and a 40 kD branched mono-methoxy PEG and PEG-INTRON® is a conjugate of interferon α-2b and a 12 kD mono-methoxy PEG. See, for example, B. A. Luxon et al., Clin. Therap. 2002, 24, 1363-1383; and A. Kozlowski and J. M. Harris, J. Control. Release, 2001, 72, 217-224.

Combination therapy of HCV with ribavirin and interferon-α currently is the optimal therapy for HCV. Combining ribavirin and PEG-IFN (infra) results in a sustained viral response in 54-56% of patients. The SVR approaches 80% for type 2 and 3 HCV. Walker, supra. Unfortunately, combination therapy also produces side effects which pose clinical challenges. Depression, flu-like symptoms and skin reactions are associated with subcutaneous IFN-α and hemolytic anemia is associated with sustained treatment with ribavirin.

A number of potential molecular targets for drug development as anti-HCV therapeutics have now been identified including, but not limited to, the NS2-NS3 autoprotease, the NS3 protease, the NS3 helicase and the NS5B polymerase. The RNA-dependent RNA polymerase is essential for replication of the single-stranded, positive sense, RNA genome. This enzyme has elicited significant interest among medicinal chemists. Both nucleoside and non-nucleoside inhibitors of NS5B polymerase have been identified.

Nucleoside inhibitors can act either as a chain terminator or as a competitive inhibitor that interferes with nucleotide binding to the polymerase. To function as a chain terminator the nucleoside analog must be taken up by the cell and converted in vivo to a triphosphate to compete for the polymerase nucleotide binding site. This conversion to the triphosphate is commonly mediated by cellular kinases which imparts additional structural limitations on any nucleoside. In addition, this limits the direct evaluation of nucleosides as inhibitors of HCV replication to cell-based assays.

Non-nucleoside allosteric inhibitors of HIV reverse transcriptase have proven effective therapeutics alone and in combination with nucleoside inhibitors and with protease inhibitors. Several classes of non-nucleoside HCV NS5B inhibitors have been described and are currently at various stages of development including: benzimidazoles, (H. Hashimoto et al. WO 01/47833, H. Hashimoto et al. WO 03/000254, P. L. Beaulieu et al. WO 03/020240 A2; P. L. Beaulieu et al. U.S. Pat. No. 6,448,281 B1; P. L. Beaulieu et al. WO 03/007945 A1); indoles, (P. L. Beaulieu et al. WO 03/0010141 A2); benzothiadiazines, e.g., 1, (D. Dhanak et al. WO 01/85172 A1, filed May 10, 2001; D. Chai et al., WO2002098424, filed Jun. 7, 2002, D. Dhanak et al WO 03/037262 A2, filed Oct. 28, 2002; K. J. Duffy et al. WO03/099801 A1, filed May 23, 2003, M. G. Darcy et al. WO2003059356, filed Oct. 28, 2002; D. Chai et al. WO 2004052312, filed Jun. 24, 2004, D. Chai et al. WO2004052313, filed Dec. 13, 2003; D. M. Fitch et al., WO2004058150, filed Dec. 11, 2003; D. K. Hutchinson et al. WO2005019191, filed Aug. 19, 2004; J. K. Pratt et al. WO 2004/041818 A1, filed Oct. 31, 2003); thiophenes, e.g., 2, (C. K. Chan et al. WO 02/100851 A2); benzothiophenes (D. C. Young and T. R. Bailey WO 00/18231); β-ketopyruvates (S. Attamura et al. U.S. Pat. No. 6,492,423 B1, A. Attamura et al. WO 00/06529); pyrimidines (C. Gardelli et al. WO 02/06246 A1); pyrimidinediones (T. R. Bailey and D. C. Young WO 00/13708); triazines (K.-H. Chung et al. WO 02/079187 A1); rhodanine derivatives (T. R. Bailey and D. C. Young WO 00/10573, J. C. Jean et al. WO 01/77091 A2); 2,4-dioxopyrans (R. A. Love et al. EP 256628 A2); phenylalanine derivatives (M. Wang et al. J. Biol. Chem. 2003 278:2489-2495).

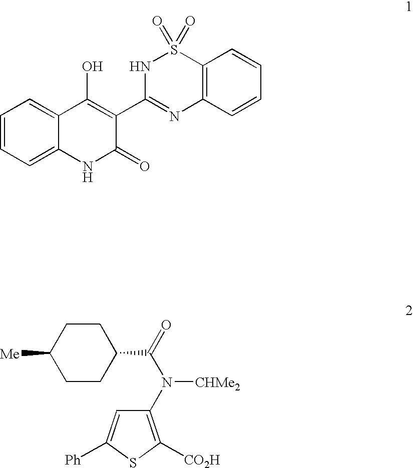

Thiazines that inhibit HCV NS5B have been disclosed by J. F. Blake et al. in U.S. Pub No. 200600040927, filed Aug. 22, 2005 and by J. B. Fell in U.S. Ser. No. 60/774,419 filed Feb. 17, 2006.

While a variety of compounds have been developed for treating HCV, there is a continuing need for new HCV treatments.

### SUMMARY OF THE INVENTION

One aspect of the invention provides a compound of the formula:

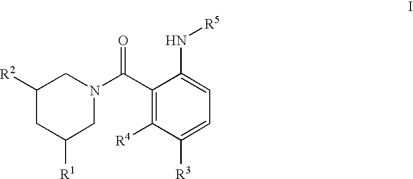

where
R¹ and R² are independently hydrogen, C₁₋₆ alkyl or C₁₋₆ haloalkyl;
R³ is hydrogen, halogen, C₁₋₆ alkyl or C₁₋₆ alkoxy;
R⁴ is hydrogen or halogen; and
R⁵ is a succinic acid moiety, a urea moiety, an amino acid moiety or a N—C₁₋₆ acyl amino acid moiety.

Another aspect of the invention provides a method for treating hepatitis C viral infection in a subject by administering to the subject in need of such treatment a compound of Formula I.

Still other aspects of the invention provide methods for making compounds of Formula I, and methods for producing pharmaceutical compositions comprising a compound of Formula I.

### DETAILED DESCRIPTION OF THE INVENTION

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

“Alkyl” means the monovalent linear or branched saturated hydrocarbon moiety, consisting solely of carbon and hydrogen atoms, having from one to twelve carbon atoms. “Lower alkyl” refers to an alkyl group of one to six carbon atoms, i.e., C₁-C₆ alkyl. Examples of alkyl groups include, but are not limited to, methyl, ethyl, propyl, isopropyl, isobutyl, sec-butyl, tert-butyl, pentyl, n-hexyl, octyl, dodecyl, and the like. “Branched alkyl” means isopropyl, isobutyl, tert-butyl.

“Alkylene” means a linear saturated divalent hydrocarbon radical of one to six carbon atoms or a branched saturated divalent hydrocarbon radical of three to six carbon atoms, e.g., methylene, ethylene, 2,2-dimethylethylene, propylene, 2-methylpropylene, butylene, pentylene, and the like.

“Aryl” means a monovalent cyclic aromatic hydrocarbon moiety consisting of a mono-, bi- or tricyclic aromatic ring. The aryl group can be optionally substituted as defined herein. Examples of aryl moieties include, but are not limited to, phenyl, naphthyl, and the like, each of which can be optionally substituted.

“Heteroalkyl” means an alkyl radical as defined herein, including a branched C₄-C₇-alkyl, wherein one, two or three hydrogen atoms have been replaced with a substituent independently selected from the group consisting of —OR^{a}, —NR^{b}R^{c}, and —S(O)ₙR^{d} (where n is an integer from 0 to 2), with the understanding that the point of attachment of the heteroalkyl radical is through a carbon atom, wherein R^{a} is hydrogen, acyl, alkyl, cycloalkyl, or cycloalkylalkyl; R^{b} and R^{c} are independently of each other hydrogen, acyl, alkyl, cycloalkyl, or cycloalkylalkyl; and when n is 0, R^{d} is hydrogen, alkyl, cycloalkyl, or cycloalkylalkyl, and when n is 1 or 2, R^{d} is alkyl, cycloalkyl, cycloalkylalkyl, amino, acylamino, monoalkylamino, or dialkylamino. Representative examples include, but are not limited to, 2-hydroxyethyl, 3-hydroxypropyl, 2-hydroxy-1-hydroxymethylethyl, 2,3-dihydroxypropyl, 1-hydroxymethylethyl, 3-hydroxybutyl, 2,3-dihydroxybutyl, 2-hydroxy-1-methylpropyl, 2-aminoethyl, 3-aminopropyl, 2-methylsulfonylethyl, aminosulfonylmethyl, aminosulfonylethyl, aminosulfonylpropyl, methylaminosulfonylmethyl, methylaminosulfonylethyl, methylaminosulfonylpropyl, and the like.

“Heteroaryl” means a monocyclic or bicyclic radical of 5 to 12 ring atoms having at least one aromatic ring containing one, two, or three ring heteroatoms independently selected from N, O, or S, the remaining ring atoms being C, with the understanding that the attachment point of the heteroaryl radical will be on an aromatic ring. The heteroaryl ring may be optionally substituted as defined herein. Examples of heteroaryl moieties include, but are not limited to, optionally substituted imidazolyl, oxazolyl, isoxazolyl, thiazolyl, isothiazolyl, oxadiazolyl, thiadiazolyl, pyrazinyl, thienyl, thiophenyl, furanyl, pyranyl, pyridinyl, pyrrolyl, pyrazolyl, pyrimidyl, quinolinyl, isoquinolinyl, benzofuryl, benzofuranyl, benzothiophenyl, benzothiopyranyl, benzimidazolyl, benzoxazolyl, benzooxadiazolyl, benzothiazolyl, benzothiadiazolyl, benzopyranyl, indolyl, isoindolyl, triazolyl, triazinyl, quinoxalinyl, purinyl, quinazolinyl, quinolizinyl, naphthyridinyl, pteridinyl, carbazolyl, azepinyl, diazepinyl, acridinyl and the like, including partially hydrogenated derivatives thereof.

The terms “halo,” “halogen,” and “halide” are used interchangeably herein and refer to a substituent fluoro, chloro, bromo, or iodo.

“Haloalkyl” means alkyl as defined herein in which one or more hydrogen has been replaced with same or different halogen. Exemplary haloalkyls include —CH₂Cl, —CH₂CF₃, —CH₂CCl₃, perfluoroalkyl (e.g., —CF₃), and the like.

“Heterocyclyl” means a monovalent saturated moiety, consisting of one to three rings, incorporating one, two, or three or four heteroatoms (chosen from nitrogen, oxygen or sulfur). The heterocyclyl ring may be optionally substituted as defined herein. Examples of heterocyclyl moieties include, but are not limited to, optionally substituted piperidinyl, piperazinyl, homopiperazinyl, azepinyl, morpholinyl, dihydrofuryl, tetrahydrofuryl, dihydropyranyl, tetrahydropyranyl, thiamorpholinyl, thiamorpholinylsulfoxide, thiamorpholinylsulfone, dihydroquinolinyl, dihydrisoquinolinyl, tetrahydroquinolinyl, tetrahydrisoquinolinyl, and the like.

The term “1-H-imidazol-4-ylmethyl” as used herein refers to a moiety of formula

(i). Compounds of formula (I) exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. The present invention encompasses all tautomeric forms of the compounds. The term “3,4-dihydro-2H-benzo[b][1,4]dioxepin-7-ylmethyl” as used herein refers to a moiety of formula (II). The term “N—C₁₋₆ acyl amino acid” refers to a C(═O)CHR²⁰NR²¹R²² wherein R²⁰ is C₁₋₆ acyl and R²¹ and R²² are as defined in claim 9.

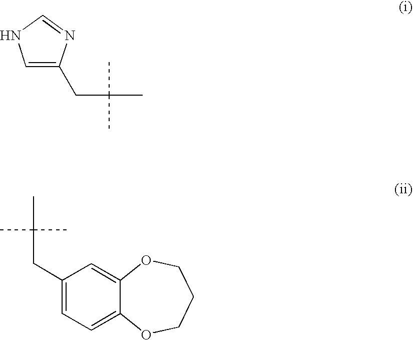

“Optionally substituted”, when used in association with “aryl”, phenyl”, “heteroaryl” or “heterocyclyl”, means an aryl, phenyl, heteroaryl or heterocyclyl which is optionally substituted independently with one or more substituents, preferably one to four, and more preferably, one to three substituents selected from alkyl, heteroalkyl, oxo (i.e., ═O), haloalkyl, —(CH₂)ₘCOX¹, —(CH₂)ₘSO₂X², alkoxy, halogen, alkylthio, alkylsulfonyl, —SO₂NR^{x}R^{y}, cyano, nitro, and —NR^{x}R^{y}, where m is an integer from 0 to 4, X¹ and X² are independently alkyl, alkoxy, amino, monoalkylamino, or dialkylamino, and R^{x} and R^{y} are independently hydrogen or alkyl.

“Leaving group” means the group with the meaning conventionally associated with it in synthetic organic chemistry, i.e., an atom or group displaceable under substitution reaction conditions. Examples of leaving groups include, but are not limited to, halogen, alkane- or arylenesulfonyloxy, such as methanesulfonyloxy, ethanesulfonyloxy, thiomethyl, benzenesulfonyloxy, tosyloxy, and thienyloxy, dihalophosphinoyloxy, optionally substituted benzyloxy, isopropyloxy, acyloxy, and the like.

“Optional” or “optionally” means that the subsequently described event or circumstance may but need not occur, and that the description includes instances where the event or circumstance occurs and instances in which it does not.

“Disease” and “Disease state” means any disease, condition, symptom, disorder or indication.

“Inert organic solvent” or “inert solvent” means the solvent is inert under the conditions of the reaction being described in conjunction therewith, including for example, benzene, toluene, acetonitrile, tetrahydrofuran, N,N-dimethylformamide, chloroform, methylene chloride or dichloromethane, dichloroethane, diethyl ether, ethyl acetate, acetone, methyl ethyl ketone, methanol, ethanol, propanol, isopropanol, tert-butanol, dioxane, pyridine, and the like. Unless specified to the contrary, the solvents used in the reactions of the present invention are inert solvents.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

“Pharmaceutically acceptable salts” of a compound means salts that are pharmaceutically acceptable, as defined herein, and that possess the desired pharmacological activity of the parent compound. Such salts include:

acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid, and the like; or

salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methylglucamine, triethanolamine, tromethamine, and the like. Acceptable inorganic bases include aluminum hydroxide, calcium hydroxide, potassium hydroxide, sodium carbonate and sodium hydroxide.

The preferred pharmaceutically acceptable salts are the salts formed from acetic acid, hydrochloric acid, sulphuric acid, methanesulfonic acid, maleic acid, phosphoric acid, tartaric acid, citric acid, sodium, potassium, calcium, zinc, and magnesium.

It should be understood that all references to pharmaceutically acceptable salts include solvent addition forms (solvates) or crystal forms (polymorphs) as defined herein, of the same acid addition sal

The terms “pro-drug” and “prodrug”, which may be used interchangeably herein, refer to any compound which releases an active parent drug according to formula I in vivo when such prodrug is administered to a mammalian subject. Prodrugs of a compound of formula I are prepared by modifying one or more functional group(s) present in the compound of formula I in such a way that the modification(s) may be cleaved in vivo to release the parent compound. Prodrugs include compounds of formula I wherein a hydroxy, amino, or sulfhydryl group in a compound of Formula I is bonded to any group that may be cleaved in vivo to regenerate the free hydroxyl, amino, or sulfhydryl group, respectively. Examples of prodrugs include, but are not limited to, esters (e.g., acetate, formate, and benzoate derivatives), carbamates (e.g., N,N-dimethylaminocarbonyl) of hydroxy functional groups in compounds of formula I, N-acyl derivatives (e.g. N-acetyl) N-Mannich bases, Schiff bases and enaminones of amino functional groups, oximes, acetals, ketals and enol esters of ketone and aldehyde functional groups in compounds of Formula I, and the like, see Bundegaard, H. “Design of Prodrugs” p 1-92, Elsevier, New York-Oxford (1985), and the like.

“Protective group” or “protecting group” means the group which selectively blocks one reactive site in a multifunctional compound such that a chemical reaction can be carried out selectively at another unprotected reactive site in the meaning conventionally associated with it in synthetic chemistry. Certain processes of this invention rely upon the protective groups to block reactive nitrogen and/or oxygen atoms present in the reactants. For example, the terms “amino-protecting group” and “nitrogen protecting group” are used interchangeably herein and refer to those organic groups intended to protect the nitrogen atom against undesirable reactions during synthetic procedures. Exemplary nitrogen protecting groups include, but are not limited to, trifluoroacetyl, acetamido, benzyl (Bn), benzyloxycarbonyl (carbobenzyloxy, CBZ), p-methoxybenzyloxycarbonyl, p-nitrobenzyloxycarbonyl, tert-butoxycarbonyl (BOC), and the like. Skilled persons will know how to choose a group for the ease of removal and for the ability to withstand the following reactions.

“Solvates” means solvent additions forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

“Subject” means mammals and non-mammals. Mammals means any member of the mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The terms “those defined above” and “those defined herein” when referring to a variable incorporates by reference the broad definition of the variable as well as preferred, more preferred and most preferred definitions, if any.

“Treating” or “treatment” of a disease state includes:
(i) preventing the disease state, i.e. causing the clinical symptoms of the disease state not to develop in a subject that may be exposed to or predisposed to the disease state, but does not yet experience or display symptoms of the disease state.
(ii) inhibiting the disease state, i.e., arresting the development of the disease state or its clinical symptoms, or
(iii) relieving the disease state, i.e., causing temporary or permanent regression of the disease state or its clinical symptoms.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

### Nomenclature and Structures

In general, the nomenclature used in this Application is based on AUTONOM™ v.4.0, a Beilstein Institute computerized system for the generation of IUPAC systematic nomenclature. Chemical structures shown herein were prepared using ISIS® version 2.4. Any open valency appearing on a carbon, oxygen or nitrogen atom in the structures herein indicates the presence of a hydrogen atom.

Whenever a chiral carbon is present in a chemical structure, it is intended that all stereoisomers associated with that chiral carbon are encompassed by the structure.

All patents and publications identified herein are incorporated herein by reference in their entirety.

### Compounds of the Invention

One aspect of the invention provides a compound of formula:

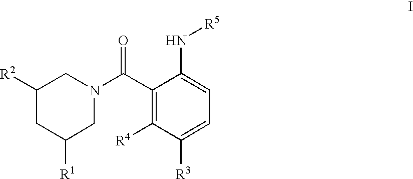

where
R¹ and R² are independently hydrogen, C₁₋₆ alkyl or C₁₋₆ haloalkyl;
R³ is hydrogen, halogen, C₁₋₆ alkyl or C₁₋₆ alkoxy;
R⁴ is hydrogen or halogen; and
R⁵ is a succinic acid moiety, a urea moiety, an amino acid moiety or a N—C₁₋₆ acyl amino acid moiety.

As used herein, the terms “a derivative thereof” refers to a compound which can be readily synthesized from the given starting material, e.g., mono- or di-amino compounds, succinic acid or succinic anhydride or succinic ester compounds, or urea compounds, and which comprises the basic subunit which resembles the stated moiety. For example, a succinic acid moiety derivative refers to a moiety that comprises —X—C(═O)—(CH₂)₂—C(═O)—X— type of frame work, where each X can be independently O or NR^{z}, where R^{z} is hydrogen or alkyl; moreover, the two X moieties can be linked through a linker to form a cyclic structure. Typically, the “derivative compound” or the “derivative moiety” can be synthesized from a readily available starting material by performing five (5) chemical reactions or less and often three (3) chemical reactions or less. Suitable chemical reactions for converting a starting material to a corresponding derivative are well known to one skilled in the art.

In some embodiments, each of R¹ and R² is independently C₁₋₆ alkyl or C₁₋₆ haloalkyl. Within these embodiments, in some instances each of R¹ and R² is independently methyl, ethyl or trifluoromethyl.

In other embodiments, R¹ and R² are in a cis-configuration relative to each other.

Yet in other embodiments, R³ is halogen, C₁₋₆ alkyl or C₁₋₆ alkoxy. Within these embodiments, in some instances, R³ is halogen. Often R³ is Br, Cl, or I.

Still in other embodiments, R⁴ is hydrogen or F.

In other embodiments, R⁵ is C(═O)CHR²⁰NR²¹R²², wherein R²⁰ is hydrogen, C₁₋₆ alkyl, heteroalkyl, C₁₋₆ heteroaralkyl, (CH₂)ₙC(═O)NH₂ wherein n is 1 or 2, CH₂OH or 4-imidazol-4-yl-methyl or a derivative thereof.

Still in other embodiments, R⁵ is selected from the group consisting of:
(i) C(═O)NR¹⁰R¹¹, wherein
   R¹⁰ is hydrogen or C₁₋₆ alkyl; and
   R¹¹ is:
      (a) aralkyl, wherein the aryl group is optionally substituted with halogen, amino, C₁₋₆ monoalkylamino, or C₁₋₆ dialkylamino,
      (b) heteroaralkyl, wherein the heteroaryl group is optionally substituted with alkyl, or
      (c) CHR¹²CONR¹³R¹⁴, wherein
         R¹² is selected from the group consisting of hydrogen or C₁₋₆ alkyl, and
         R¹³ and R¹⁴ are independently hydrogen or C₁₋₆ alkyl,
         or R¹³ and R¹⁴ taken together are (CH₂)₂O(CH₂)₂;
      or R¹⁰ and R¹¹ taken together along with the nitrogen atom to which they are attached form a pyrrolidine moiety of formula (I) wherein each of R¹⁶ and R¹⁷ is independently hydrogen or C₁₋₆ alkyl, or, R¹⁶ and R¹⁷ taken together are (CH₂)₂—O—(CH₂)₂—;

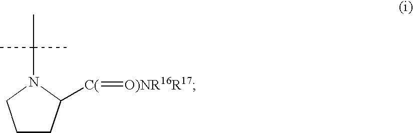

(ii) C(═O)CHR²⁰NR²¹R²², wherein
   R²⁰ is hydrogen, C₁₋₆ alkyl, heteroalkyl, or C₁₋₆ heteroaralkyl, (CH₂)ₙC(═O)NH₂ wherein n is 1 or 2, CH₂OH or 4-imidazol-4-yl-methyl;
   R²¹ is hydrogen or C₁₋₆ alkyl, typically hydrogen;
   R²² is —C(═O)R²³, wherein R²³ is alkyl, heteroaryl (which is optionally substituted with one or two substituents each of which is independently selected from the group consisting of C₁₋₆ alkyl, alkoxy, halogen, and morpholin-4-yl), C₁₋₆heteroaralkyl, amino, monoalkylamino, dialkylamino, aryl (which is optionally substituted with one or two substituents each of which is independently selected from the group consisting of alkyl and halogen, typically aryl is optionally substituted with halogen), aralkyl, alkoxy, aralkoxy, heteroalkyl, tetrahydrofuran-2-yl, or heterocyclylalkyl; or
   R²⁰ and R²¹ together are (CH₂)₃, CH₂CH(OH)CH₂, or a moiety of the formula:

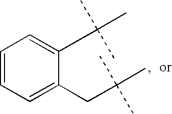

R²¹ and R²² together are C(═O)OCH₂CH₂ or CH₂CH₂OCH₂CH₂, or
R²⁰ and R²² together are C(═O)NR²⁴C(═O)CH₂, wherein R²⁴ is hydrogen or alkyl; and
(iii) C(═O)CHR³⁰CHR³¹COR³², wherein
   R³⁰ and R³¹ are hydrogen; and
   R³² is OR⁴⁰, NR³³R³⁴, alkyl, optionally substituted phenyl, 3,4-dihydro-2H-benzo[b][1,4]dioxepin-7-ylmethyl, or heteroaryl;
   wherein
      each of R³³ and R⁴⁰ is independently hydrogen or C₁₋₆ alkyl,
      R³⁴ is hydrogen, C₁₋₆ alkyl, optionally substituted C₁₋₆ aralkyl, C₁₋₆ heteroaralkyl, or heteroaryl; or
   R³⁰ and R³¹ together are —(CH₂)ₙ—, wherein n is 1, 2, 3, or 4 (in some instances n is 4); or
   R³⁰ and R³³ together are —CH₂—; or
   R³⁰ and R³² together are optionally substituted 1,2-phenylene, —H₂—CH₂—; or
   R³³ and R³⁴ together are —CH₂CHR⁴¹—X—CHR⁴²CH₂—, —(CH₂)₅—, —CH₂CHR³⁶—(CH₂)₃—;
   X is NR³⁵, O or S;
   R³⁵ is hydrogen, C₁₋₆ alkyl, or a nitrogen C₁₋₆ acyl;
   each of R⁴¹ and R⁴² is independently hydrogen or C₁₋₆ alkyl;
   R³⁶ is C(═O)NR³⁷R³⁸; and
   each of R³⁷ and R³⁸ is independently hydrogen or C₁₋₆ alkyl.

In some embodiments, R⁵ is a urea or a derivative thereof. Within these embodiments, typically R⁵ is a moiety of the formula C(═O)NR¹⁰R¹¹, where R¹⁰ and R¹¹ are those defined herein. In some cases, R¹⁰ is hydrogen or methyl. In other cases, R¹¹ is aralkyl. Often the aryl group of R¹¹ is phenyl which is optionally substituted with one or two substituents each of which is independently selected from the group consisting of halogen, amino, monoalkylamino, and dialkylamino. Still in other cases, R¹¹ is heteroaralkyl. Typically the heteroaryl group of R¹¹ is selected from the group consisting of: pyridinyl; 1,5-dimethyl-1H-pyrazol-3-yl; 1-methyl-1H-pyrrol-2-yl; 5-methyl-isoxazol-3-yl; and 5-methyl-pyrazin-2-yl. Yet in other instances, R¹¹ is selected from the group consisting of benzyl, (1-methyl-1H-pyrrol-3-yl)methyl, (5-methyl-isoxazol-3-yl)methyl, (1,5-dimethyl-1H-pyrazol-3-yl)methyl, (pyridin-2-yl)methyl, (pyridin-3-yl)methyl, (pyridin-4-yl)methyl, 2-fluorophenylmethyl, 4-(N,N-dimethylamino)phenylmethyl, and a moiety of the formula: CHR¹²CONR¹³R¹⁴, wherein R¹² is hydrogen or methyl, and R¹³ and R¹⁴ are methyl or R¹³ and R¹⁴ together are —(CH₂)₂—O—(CH₂)₂—. Yet still in other instances, R¹⁰ and R¹¹ taken together along with the nitrogen atom to which they are attached form a pyrrolidine moiety of the formula:

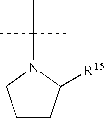

wherein
   R¹⁵ is C(═O)NR¹⁶R¹⁷,
   and wherein
      each of R¹⁶ and R¹⁷ is independently hydrogen or alkyl,
      or R¹⁶ and R¹⁷ taken together are —(CH₂)₂—O(CH₂)₂—.

Still in other embodiments, R⁵ is an amino acid or a derivative thereof. Within these embodiments, R⁵ is typically a moiety of the formula: C(═O)CHR²⁰NR²¹R²², where R²⁰, R²¹, and R²² are those defined herein. Within these embodiments, in some instances R²⁰ is hydrogen, methyl, ethyl, hydroxymethyl, 1H-imidazol-4-ylmethyl, or a heteroalkyl of the formula: —(CH₂)ₙC(═O)NR²⁵R²⁶, where n is 1 or 2, and each of R²⁵ and R²⁶ is independently hydrogen or alkyl. Still in other instances, R²¹ is hydrogen. Yet in other instances, R²² is a moiety of the formula —C(═O)R²³, where R²³ is methyl, pyridin-2-yl, 6-methylpyridin-2-yl, pyridin-3-yl, furan-2-yl, phenyl, benzyl, amino, iso-butyl, methoxy, 2-(morpholin-4-yl)pyridin-3-yl, benzyloxy, 4-fluorophenyl, tert-butoxy, ethyl, 2,6-difluorophenyl, thiophen-2-yl, thiophen-2-ylmethyl, pyrazin-2-yl, 1-methyl-1H-pyrrol-2-yl, pyridin-4-yl, tetrahydrofuran-2-yl, 3H-imidazol-4-yl, isoxazol-5-yl, furazan-3-yl, 5-methyl-isoxazol-3-yl, 1-methyl-1H-imidazol-2-yl, (2-oxo-oxazolidin-3-yl)methyl, 6-methyl-2-oxo-1,2-dihydropyrimidin-4-yl, 5-methylfuran-2-yl, 5-ethoxyfuran-2-yl, or 2-chloropyridin-3-yl.

Yet in other embodiments, R⁵ is a succinic acid moiety or a derivative thereof. Within these embodiments, R⁵ is typically a moiety of the formula: C(═O)CHR³⁰CHR³¹COR³², wherein R³⁰, R³¹ and R³² are those defined herein.

Still yet in other embodiments, compounds of Formula I are compounds represented by the following formula:

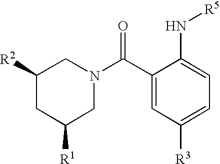

where
   each of R¹ and R² is independently methyl or trifluoromethyl;
   R³ is halogen; and
   R⁵ is as defined herein.

It should be appreciated that combinations of the different groups described herein may form other embodiments. In this manner, a variety of different compounds are embodied within the present invention.

Representative compounds of the invention are shown in Table 1 below.

TABLE 1
Representative compounds of Formula I.

| | | | | | IC₅₀ |
|---|---|---|---|---|---|
| Cpd. | | | ms | | HCV |
| No. | Structure | mw | (M + H)⁺ | Name | pol¹ |
| I-1 | 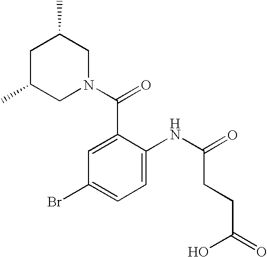 | | 411 | N-[4-Bromo-2-((3S,5R)-3,5-dimethyl-piperidine-1-carbonyl)-phenyl]-succinamic acid | C |
| I-2 | 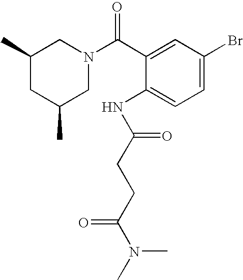 | 438.36 | 438 | N-[4-Bromo-2-((3S,5R)-3,5-dimethyl-piperidine-1-carbonyl)-phenyl]-N′,N′-dimethyl-succinamide | A |
| I-3 | 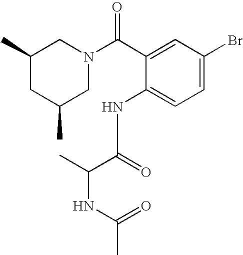 | 424.34 | 424 | 2-Acetylamino-N-[4-bromo-2-((3R,5S)-3,5-dimethyl-piperidine-1-carbonyl)-phenyl]-propionamide | C |
| I-4 | 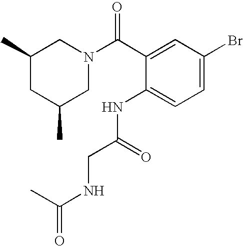 | 410.31 | 410 | 2-Acetylamino-N-[4-bromo-2-((3R,5S)-3,5-dimethyl-piperidine-1-carbonyl)-phenyl]-acetamide | C |
| I-5 | 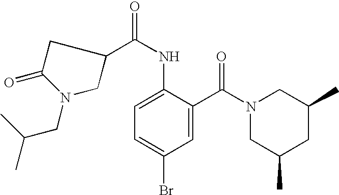 | 478.43 | 478 | 1-Isobutyl-5-oxo-pyrrolidine-3-carboxylicacid [4-bromo-2-((3R,5S)-3,5-dimethyl-piperidine-1-carbonyl)-phenyl]-amide | C |
| I-6 | 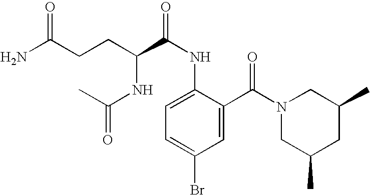 | 481.39 | 481 | (S)-2-Acetylamino-pentanedioic acid 5-amide1-{[4-bromo-2-((3S,5R)-3,5-dimethyl-piperidine-1-carbonyl)-phenyl]-amide} | B |
| I-7 | 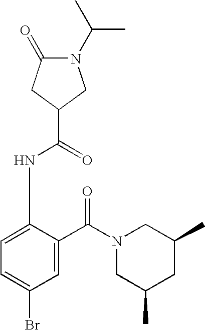 | 464.4 | 464 | 1-Isopropyl-5-oxo-pyrrolidine-3-carboxylicacid [4-bromo-2-((3R,5S)-3,5-dimethyl-piperidine-1-carbonyl)-phenyl]-amide | C |
| I-8 | 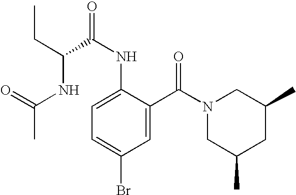 | 438.36 | 438 | (R)-2-Acetylamino-N-[4-bromo-2-((3R,5S)-3,5-dimethyl-piperidine-1-carbonyl)-phenyl]-butyramide | C |
| I-9 | 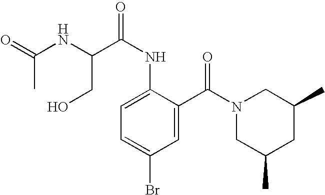 | 440.34 | 440 | 2-Acetylamino-N-[4-bromo-2-((3S,5R)-3,5-dimethyl-piperidine-1-carbonyl)-phenyl]-3-hydroxy-propionamide | C |
| I-10 | 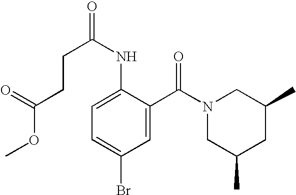 | 425.32 | 425 | N-[4-Bromo-2-((3R,5S)-3,5-dimethyl-piperidine-1-carbonyl)-phenyl]-succinamic acid methylester | C |
| I-11 | 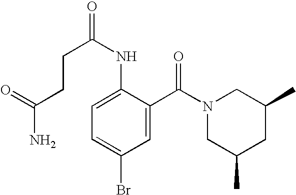 | 410.31 | 410 | N-[4-Bromo-2-((3R,5S)-3,5-dimethyl-piperidine-1-carbonyl)-phenyl]-succinamide | C |
| I-12 | 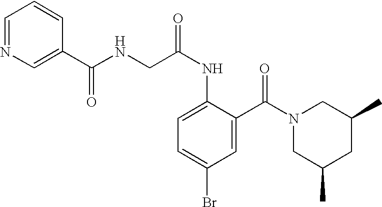 | 473.37 | 473 | N-{[4-Bromo-2-((3R,5S)-3,5-dimethyl-piperidine-1-carbonyl)-phenylcarbamoyl]-methyl}-nicotinamide | B |
| I-13 | 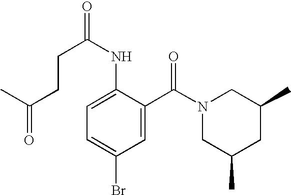 | 409.32 | 409 | 4-Oxo-pentanoic acid [4-bromo-2-((3R,5S)-3,5-dimethyl-piperidine-1-carbonyl)-phenyl]-amide | C |
| I-14 | 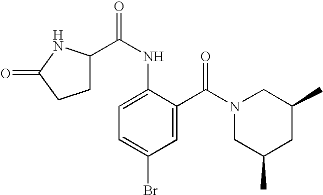 | 422.32 | 422 | 5-Oxo-pyrrolidine-2-carboxylic acid [4-bromo-2-((3R,5S)-3,5-dimethyl-piperidine-1-carbonyl)-phenyl]-amide | C |
| I-15 | | 480.4 | 480 | N-[4-Bromo-2-((3R,5S)- | A |
| | | | | 3,5-dimethyl-piperidine-1- | |
| | | | | carbonyl)-phenyl]-4- | |
| | | | | morpholin-4-yl-4-oxo- | |
| | | | | butyramide | |
| I-16 | 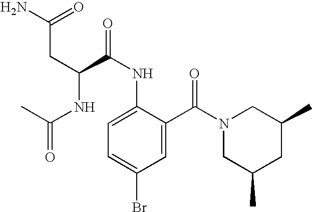 | 467.36 | 467 | (S)-2-Acetylamino-N1-[4-bromo-2-((3S,5R)-3,5-dimethyl-piperidine-1-carbonyl)-phenyl]-succinamide | C |
| I-17 | 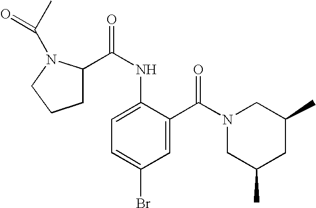 | 450.37 | 450 | 1-Acetyl-pyrrolidine-2-carboxylic acid [4-bromo-2-((3R,5S)-3,5-dimethyl-piperidine-1-carbonyl)-phenyl]-amide | C |
| I-18 | 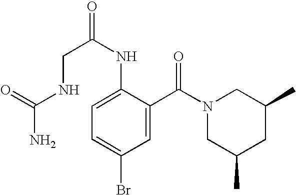 | 411.3 | 411 | N-[4-Bromo-2-((3S,5R)-3,5-dimethyl-piperidine-1-carbonyl)-phenyl]-2-ureido-acetamide | C |
| I-19 | 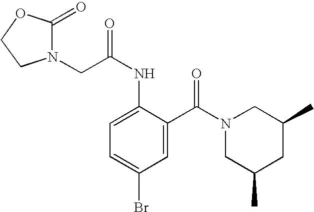 | 438.32 | 438 | N-[4-Bromo-2-((3S,5R)-3,5-dimethyl-piperidine-1-carbonyl)-phenyl]-2-(2-oxo-oxazolidin-3-yl)-acetamide | C |
| I-20 | 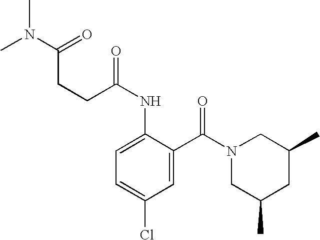 | 393.91 | 394 | N-[4-Chloro-2-((3S,5R)-3,5-dimethyl-piperidine-1-carbonyl)-phenyl]-N′,N′-dimethyl-succinamide | C |
| I-21 | 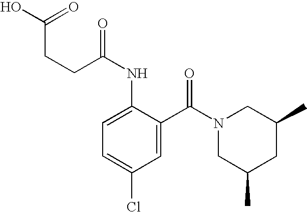 | 366.84 | 367 | N-[4-Chloro-2-((3R,5S)-3,5-dimethyl-piperidine-1-carbonyl)-phenyl]-succinamide acid | C |
| I-22 | 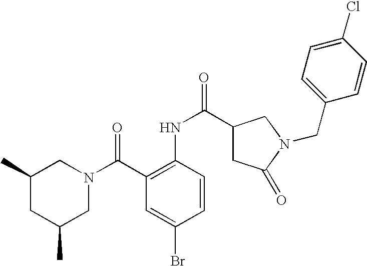 | 546.89 | 546 | 1-(4-Chloro-benzyl)-5-oxo-pyrrolidine-3-carboxylicacid [4-bromo-2-((3R,5S)-3,5-dimethyl-piperidine-1-carbonyl)-phenyl]-amide | C |
| I-23 | 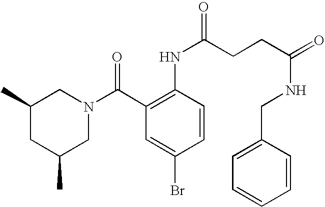 | 500.43 | 500 | N-Benzyl-N′-[4-bromo-2-((3R,5S)-3,5-dimethyl-piperidine-1-carbonyl)-phenyl]-succinamide | C |
| I-24 | 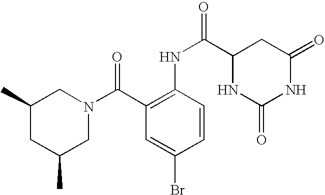 | 451.32 | 451 | 2,6-Dioxo-hexahydro-pyrimidine-4-carboxylicacid [4-bromo-2-((3S,5R)-3,5-dimethyl-piperidine-1-carbonyl)-phenyl]-amide | C |
| I-25 | 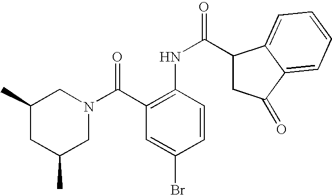 | 469.38 | 469 | 3-Oxo-indan-1-carboxylicacid [4-bromo-2-((3R,5S)-3,5-dimethyl-piperidine-1-carbonyl)-phenyl]-amide | C |
| I-26 | 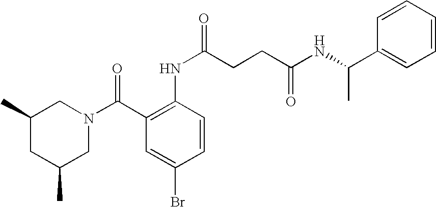 | 514.46 | 514 | N-[4-Bromo-2-((3S,5R)-3,5-dimethyl-piperidine-1-carbonyl)-phenyl]-N′-((S)-1-phenyl-ethyl)-succinamide | C |
| I-27 | 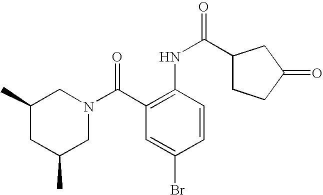 | 421.33 | 421 | 3-Oxo-cyclopentanecarboxylicacid [4-bromo-2-((3R,5S)-3,5-dimethyl-piperidine-1-carbonyl)-phenyl]-amide | C |
| I-28 | 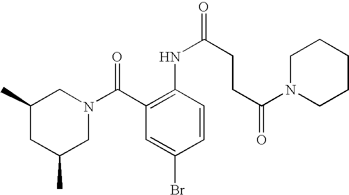 | 478.43 | 478 | N-[4-Bromo-2-((3R,5S)-3,5-dimethyl-piperidine-1-carbonyl)-phenyl]-4-oxo-4-piperidin-1-yl-butyramide | C |
| I-29 | 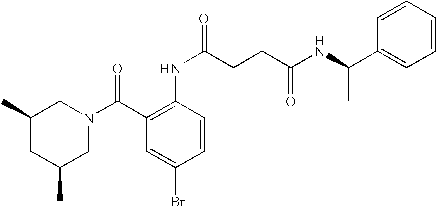 | 514.46 | 514 | N-[4-Bromo-2-((3S,5R)-3,5-dimethyl-piperidine-1-carbonyl)-phenyl]-N′-((R)-1-phenyl-ethyl)-succinamide | C |
| I-30 | 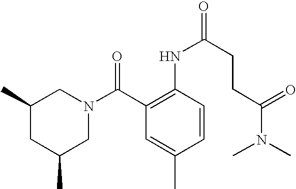 | 373.49 | 374 | N-[2-((3S,5R)-3,5-Dimethyl-piperidine-1-carbonyl)-4-methyl-phenyl]-N′,N′-dimethyl-succinamide | C |
| I-31 | 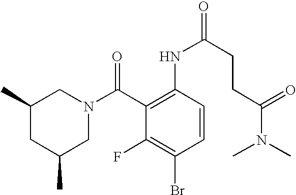 | 456.35 | 456 | N-[4-Bromo-((3R,5S)-3,5-dimethyl-piperidine-1-carbonyl)-3-fluoro-phenyl]-N′,N′-dimethyl-succinamide | C |
| I-32 | 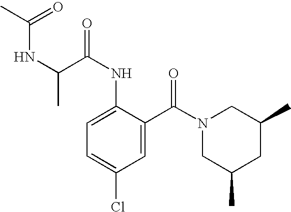 | 379.89 | 380 | 2-Acetylamino-N-[4-chloro-2-((3S,5R)-3,5-dimethyl-piperidine-1-carbonyl)-phenyl]-propionamide | C |
| I-33 | 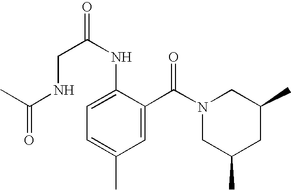 | 345.44 | 346 | 2-Acetylamino-N-[2-((3R,5S)-3,5-dimethyl-piperidine-1-carbonyl)-4-methyl-phenyl]-acetamide | C |
| I-34 | 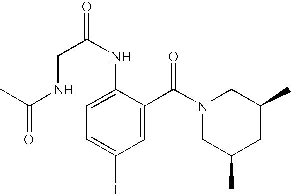 | 457.31 | 458 | 2-Acetylamino-N-[2-((3R,5S)-3,5-dimethyl-piperidine-1-carbonyl)-4-iodo-phenyl]-acetamide | C |
| I-35 | 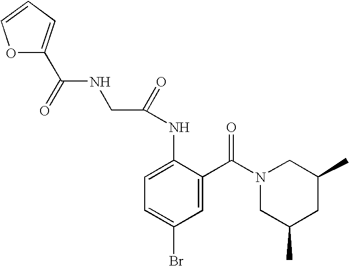 | 462.34 | 462 | Furan-2-carboxylic acid{[4-bromo-2-((3S,5R)-3,5-dimethyl-piperidine-1-carbonyl)-phenylcarbamoyl]-methyl}-amide | A |
| I-36 | 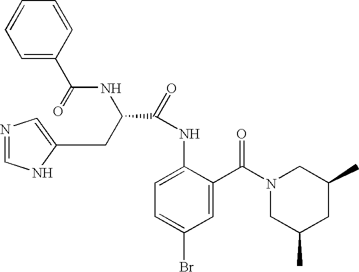 | 552.47 | 552 | N-[(S)-1-[4-Bromo-2-((3S,5R)-3,5-dimethyl-piperidine-1-carbonyl)-phenylcarbamoyl]-2-(3H-imidazol-4-yl)-ethyl]-benzamide | B |
| I-37 | 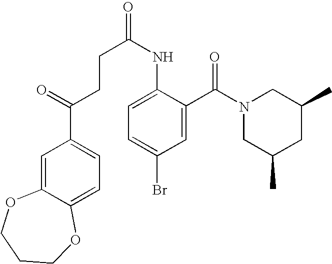 | 543.46 | 543 | N-[4-Bromo-2-((3S,5R)-3,5-dimethyl-piperidine-1-carbonyl)-phenyl]-4-(3,4-dihydro-2H-benzo[b][1,4]dioxepin-7-yl)-4-oxo-butyramide | C |
| I-38 | 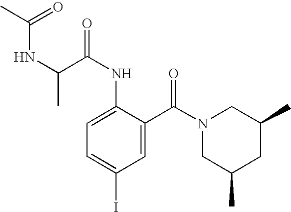 | 471.33 | 472 | 2-Acetylamino-N-[2-((3R,5S)-3,5-dimethyl-piperidine-1-carbonyl)-4-iodo-phenyl]-propionamide | C |
| I-39 | 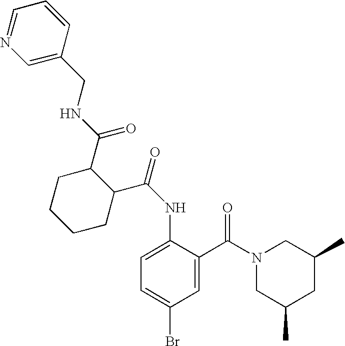 | 555.51 | 555 | Cyclohexane-1,2-dicarboxylic acid 1-{[4-bromo-2-((3R,5S)-3,5-dimethyl-piperidine-1-carbonyl)-phenyl]-amide}2-[(pyridin-3-ylmethyl)-amide] | C |
| I-40 | 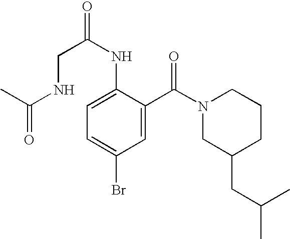 | 438.36 | 438 | 2-Acetylamino-N-[4-bromo-2-(3-isobutyl-piperidine-1-carbonyl)-phenyl]-acetamide | C |
| I-41 | 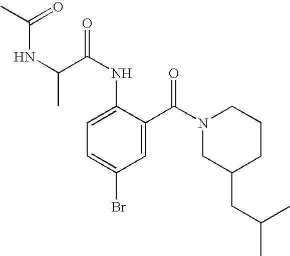 | 452.39 | 452 | 2-Acetylamino-N-[4-bromo-2-(3-isobutyl-piperidine-1-carbonyl)-phenyl]-propionamide | C |
| I-42 | 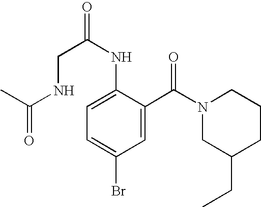 | 410.31 | 410 | 2-Acetylamino-N-[4-bromo-2-(3-ethyl-piperidine-1-carbonyl)-phenyl]-acetamide | C |
| I-43 | 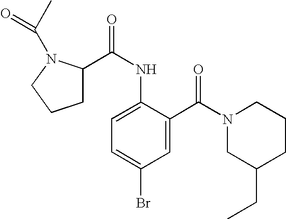 | 450.37 | 450 | 1-Acetyl-pyrrolidine-2-carboxylic acid [4-bromo-2-(3-ethyl-piperidine-1-carbonyl)-phenyl]-amide | C |
| I-44 | 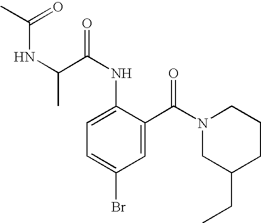 | 424.34 | 424 | 2-Acetylamino-N-[4-bromo-2-(3-ethyl-piperidine-1-carbonyl)-phenyl]-propionamide | C |
| I-45 | 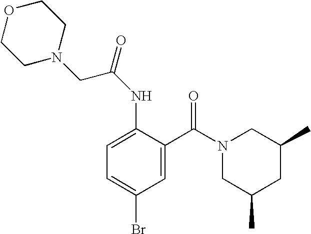 | 438.36 | 438 | N-[4-Bromo-2-((3R,5S)-3,5-dimethyl-piperidine-1-carbonyl)-phenyl]-2-morpholin-4-yl-acetamide | C |
| I-46 | 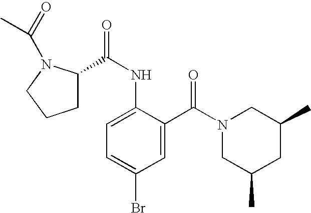 | 450.37 | 450 | (S)-1-Acetyl-pyrrolidine-2-carboxylic acid [4-bromo-2-((3S,5R)-3,5-dimethyl-piperidine-1-carbonyl)-phenyl]-amide | C |
| I-47 | 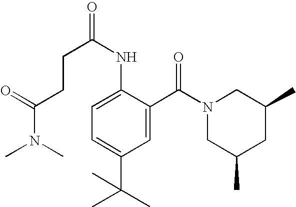 | 415.57 | 416 | N-[4-tert-Butyl-2-((3S,5R)-3,5-dimethyl-piperidine-1-carbonyl)-phenyl]-N′,N′-dimethyl-succinamide | C |
| I-48 | 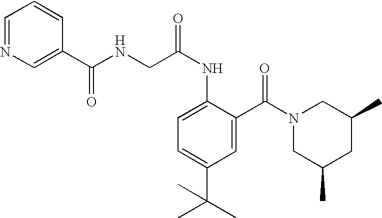 | 450.58 | 451 | N-{[4-tert-Butyl-2-((3S,5R)-3,5-dimethyl-piperidine-1-carbonyl)-phenylcarbamoyl]-methyl}-nicotinamide | C |
| I-49 | 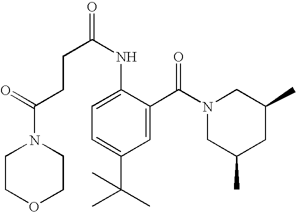 | 457.61 | 458 | N-[4-tert-Butyl-2-((3S,5R)-3,5-dimethyl-piperidine-1-carbonyl)-phenyl]-4-morpholin-4-yl-4-oxo-butyramide | C |
| I-50 | 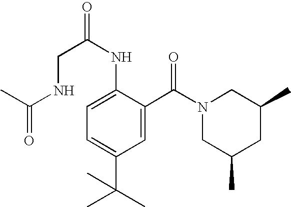 | 387.52 | 388 | 2-Acetylamino-N-[4-tert-butyl-2-((3S,5R)-3,5-dimethyl-piperidine-1-carbonyl)-phenyl]-acetamide | C |
| I-51 | I-1 | 428.92 | 429 | N-{[4-Chloro-2-((3S,5R)- | B |
| | | | | 3,5-dimethyl-piperidine-1- | |
| | | | | carbonyl)- | |
| | | | | phenylcarbamoyl]-methyl}- | |
| | | | | nicotinamide | |
| I-52 | 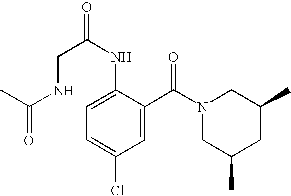 | 365.86 | 366 | 2-Acetylamino-N-[4-chloro-2-((3S,5R)-3,5-dimethyl-piperidine-1-carbonyl)-phenyl]-acetamide | C |
| I-53 | 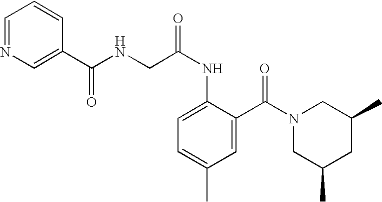 | 408.5 | 409 | N-{[2-((3S,5R)-3,5-Dimethyl-piperidine-1-carbonyl)-4-methyl-phenylcarbamoyl]-methyl}-nicotinamide | C |
| I-54 | 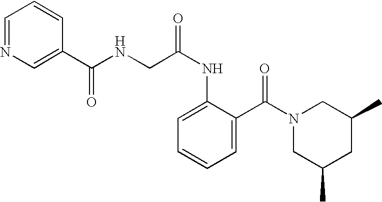 | 394.47 | 395 | N-{[2-((3S,5R)-3,5-Dimethyl-piperidine-1-carbonyl)-phenylcarbamoyl]-methyl}-nicotinamide | C |
| I-55 | 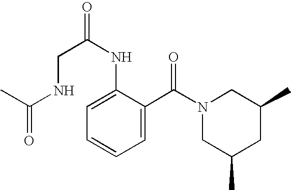 | 331.41 | 332 | 2-Acetylamino-N-[2-((3S,5R)-3,5-dimethyl-piperidine-1-carbonyl)-phenyl]-acetamide | C |
| I-56 | 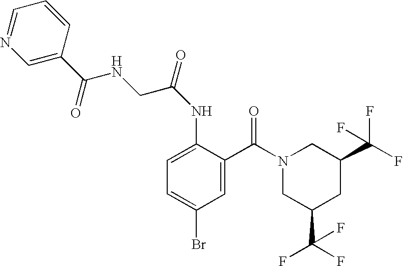 | 581.31 | 581 | N-{[2-((3R,5S)-3,5-Bis-trifluoromethyl-piperidine-1-carbonyl)-4-bromo-phenylcarbamoyl]-methyl}-nicotinamide | C |
| I-57 | 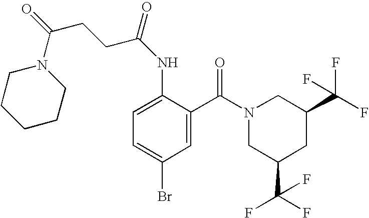 | 586.37 | 586 | N-[2-((3R,5S)-3,5-Bis-trifluoromethyl-piperidine-1-carbonyl)-4-bromo-phenyl]-4-oxo-4-piperidin-1-yl-butyramide | C |
| I-58 | 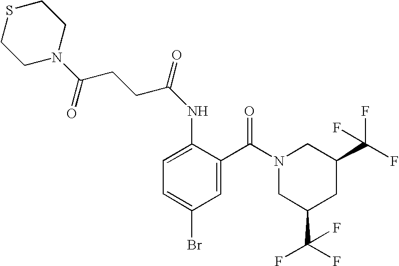 | 604.41 | 604 | N-[2-((3R,5S)-3,5-Bis-trifluoromethyl-piperidine-1-carbonyl)-4-bromo-phenyl]-4-oxo-4-thiomorpholin-4-yl-butyramide | C |
| I-59 | 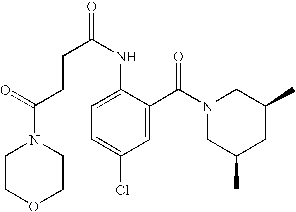 | 435.95 | 436 | N-[4-Chloro-2-((3S,5R)-3,5-dimethyl-piperidine-1-carbonyl)-phenyl]-4-morpholin-4-yl-4-oxo-butyramide | C |
| I-60 | 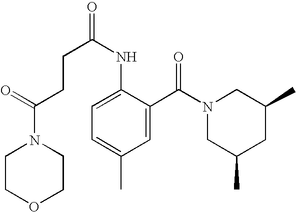 | 415.53 | 416 | N-[2-((3S,5R)-3,5-Dimethyl-piperidine-1-carbonyl)-4-methyl-phenyl]-4-morpholin-4-yl-4-oxo-butyramide | C |
| I-61 | 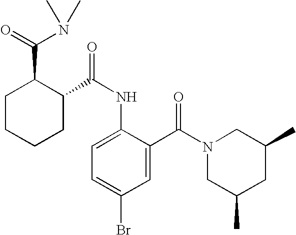 | 492.46 | 492 | (1R,2R)-Cyclohexane-1,2-dicarboxylic acid 1-{[4-bromo-2-((3R,5S)-3,5-dimethyl-piperidine-1-carbonyl)-phenyl]-amide}2-dimethylamide | C |
| I-62 | 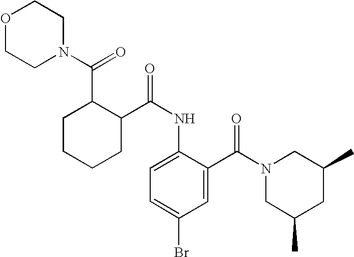 | 534.49 | 534 | 2-(Morpholine-4-carbonyl)-cyclohexanecarboxylic acid[4-bromo-2-((3S,5R)-3,5-dimethyl-piperidine-1-carbonyl)-phenyl]-amide | C |
| I-63 | 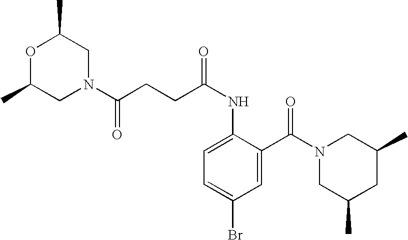 | 508.45 | 508 | N-[4-Bromo-2-((3R,5S)-3,5-dimethyl-piperidine-1-carbonyl)-phenyl]-4-((2S,6R)-dimethyl-morpholin-4-yl)-4-oxo-butyramide | C |
| I-64 | 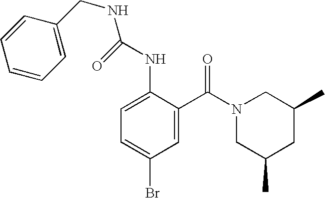 | 444.37 | 444 | 1-Benzyl-3-[4-bromo-2-((3S,5R)-3,5-dimethyl-piperidine-1-carbonyl)-phenyl]-urea | C |
| I-65 | 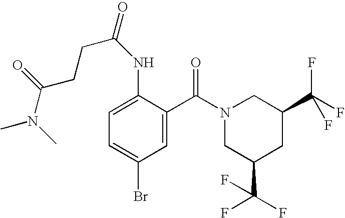 | 546.3 | 546 | N-[2-((3S,5R)-3,5-Bis-trifluoromethyl-piperidine-1-carbonyl)-4-bromo-phenyl]-N′,N′-dimethyl-succinamide | C |
| I-66 | 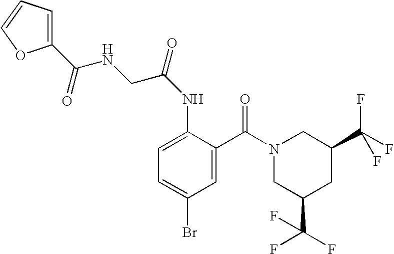 | 570.28 | 570 | Furan-2-carboxylic acid{[2-((3S,5R)-3,5-bis-trifluoromethyl-piperidine-1-carbonyl)-4-bromo-phenylcarbamoyl]-methyl}-amide | C |
| I-67 | 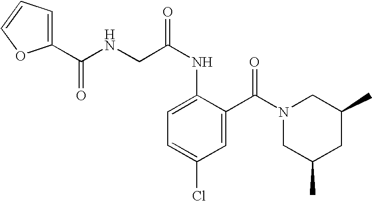 | 417.89 | 418 | Furan-2-carboxylic acid{[4-chloro-2-((3R,5S)-3,5-dimethyl-piperidine-1-carbonyl)-phenylcarbamoyl]-methyl}-amide | C |
| I-68 | 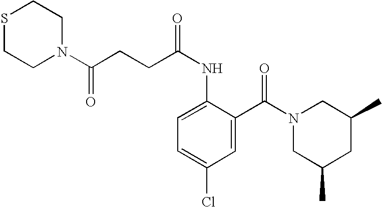 | 452.02 | #N/A | N-[4-Chloro-2-((3R,5S)-3,5-dimethyl-piperidine-1-carbonyl)-phenyl]-4-oxo-4-thiomorpholin-4-ylbutyramide | C |
| I-69 | 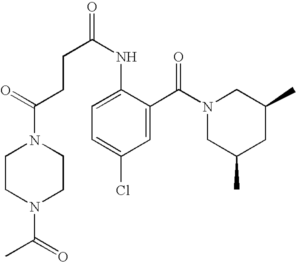 | 477 | 477 | 4-(4-Acetyl-piperazin-1-yl)-N-[4-chloro-2-((3R,5S)-3,5-dimethyl-piperidine-1-carbonyl)-phenyl]-4-oxo-butyramide | B |
| I-70 | 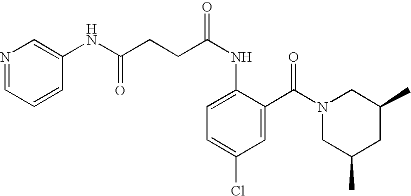 | 442.94 | 443 | N-[4-Chloro-2-((3R,5S)-3,5-dimethyl-piperidine-1-carbonyl)-phenyl]-N′-pyridin-3-yl-succinamide | C |
| I-71 | 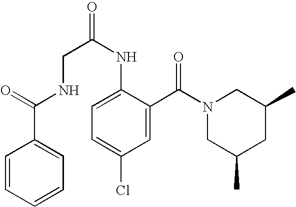 | 427.93 | 428 | N-{[4-Chloro-2-((3S,5R)-3,5-dimethyl-piperidine-1-carbonyl)-phenylcarbamoyl]-methyl}-benzamide | B |
| I-72 | 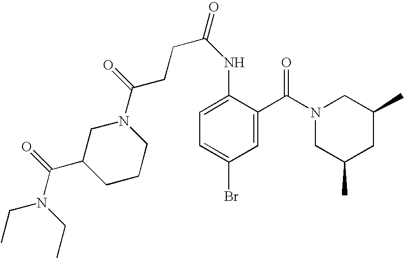 | 577.56 | 577 | 1-{3-[4-Bromo-2-((3S,5R)-3,5-dimethyl-piperidine-1-carbonyl)-phenylcarbamoyl]-propionyl}-piperidine-3-carboxylic aciddiethylamide | B |
| I-73 | 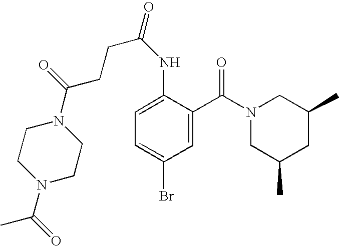 | 521.45 | 521 | 4-(4-Acetyl-piperazin-1-yl)-N-[4-bromo-2-((3R,5S)-3,5-dimethyl-piperidine-1-carbonyl)-phenyl]-4-oxo-butyramide | B |
| I-74 | 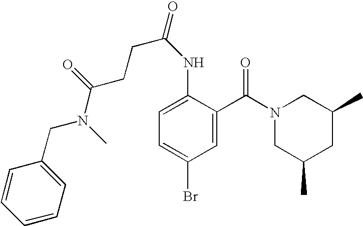 | 514.46 | 514 | N-Benzyl-N′-[4-bromo-2-((3R,5S)-3,5-dimethyl-piperidine-1-carbonyl)-phenyl]-N-methyl-succinamide | A |
| I-75 | 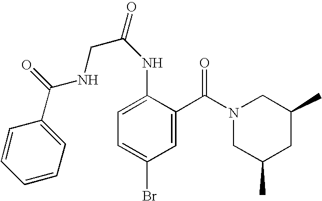 | 472.38 | 472 | N-{[4-Bromo-2-((3S,5R)-3,5-dimethyl-piperidine-1-carbonyl)-phenylcarbamoyl]-methyl}-benzamide | B |
| I-76 | 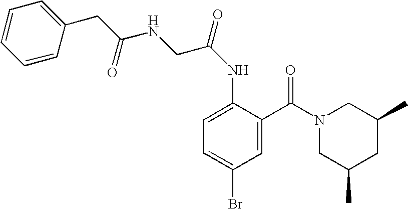 | 486.41 | 486 | N-{[4-Bromo-2-((3R,5S)-3,5-dimethyl-piperidine-1-carbonyl)-phenylcarbamoyl]-methyl}-2-phenyl-acetamide | C |
| I-77 | 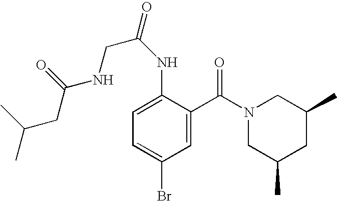 | 452.39 | 452 | N-{[4-Bromo-2-((3R,5S)-3,5-dimethyl-piperidine-1-carbonyl)-phenylcarbamoyl]-methyl}-3-methyl-butyramide | C |
| I-78 | 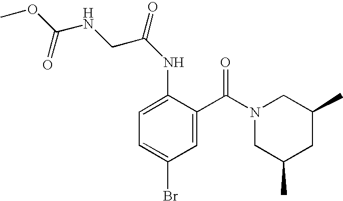 | 426.31 | 426 | {[4-Bromo-2-((3S,5R)-3,5-dimethyl-piperidine-1-carbonyl)-phenylcarbamoyl]-methyl}-carbamic acid methyl ester | C |
| I-79 | 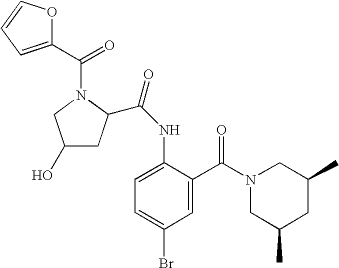 | 518.41 | 518 | 1-(Furan-2-carbonyl)-4-hydroxy-pyrrolidine-2-carboxylic acid [4-bromo-2-((3R,5S)-3,5-dimethyl-piperidine-1-carbonyl)-phenyl]-amide | C |
| I-80 | 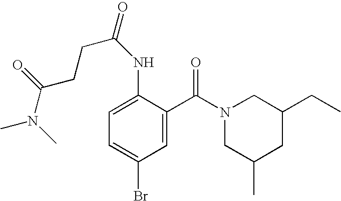 | 452.39 | 452 | N-[4-Bromo-2-(3-ethyl-5-methyl-piperidine-1-carbonyl)-phenyl]-N′,N′-dimethyl-succinamide | B |
| I-81 | 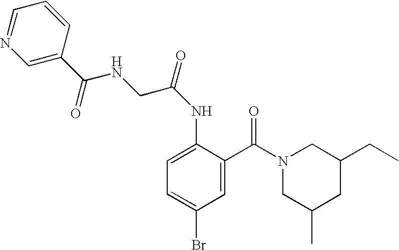 | 487.4 | 487 | N-{[4-Bromo-2-(3-ethyl-5-methyl-piperidine-1-carbonyl)-phenylcarbamoyl]-methyl}-nicotinamide | B |
| I-82 | 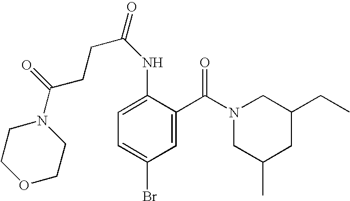 | 494.43 | 494 | N-[4-Bromo-2-(3-ethyl-5-methyl-piperidine-1-carbonyl)-phenyl]-4-morpholin-4-yl-4-oxo-butyramide | B |
| I-83 | 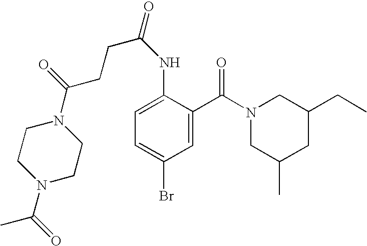 | 535.48 | 535 | 4-(4-Acetyl-piperazin-1-yl)-N-[4-bromo-2-(3-ethyl-5-methyl-piperidine-1-carbonyl)-phenyl]-4-oxo-butyramide | B |
| I-84 | 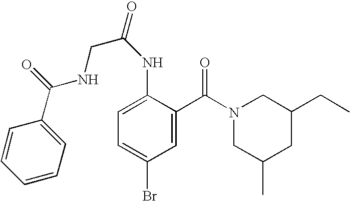 | 486.41 | 486 | N-{[4-Bromo-2-(3-ethyl-5-methyl-piperidine-1-carbonyl)-phenylcarbamoyl]-methyl}-benzamide | C |
| I-85 | 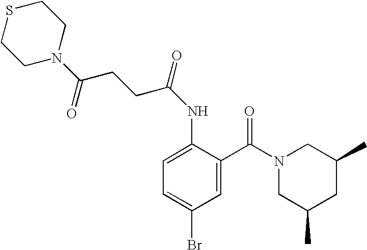 | 496.41 | 496 | N-[4-Bromo-2-((3R,5S)-3,5-dimethyl-piperidine-1-carbonyl)-phenyl]-4-oxo-4-thiomorpholin-4-yl-butyramide | C |
| I-86 | 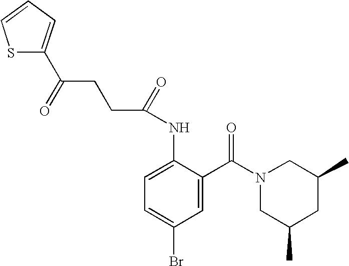 | 477.42 | 477 | N-[4-Bromo-2-((3S,5R)-3,5-dimethyl-piperidine-1-carbonyl)-phenyl]-4-oxo-4-thiophen-2-yl-butyramide | A |
| I-87 | 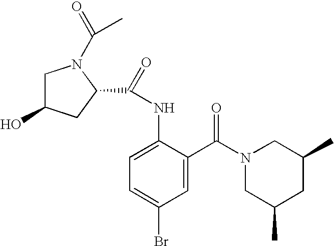 | 466.37 | 466 | (2S,4R)-1-Acetyl-4-hydroxy-pyrrolidine-2-carboxylic acid [4-bromo-2-((3R,5S)-3,5-dimethyl-piperidine-1-carbonyl)-phenyl]-amide | B |
| I-88 | 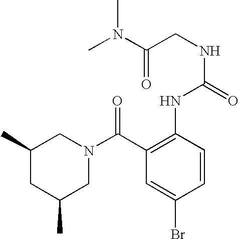 | 439.35 | 439 | 2-{3-[4-Bromo-2-((3R,5S)-3,5-dimethyl-piperidine-1-carbonyl)-phenyl]-ureido}-N,N-dimethyl-acetamide | B |
| I-89 | 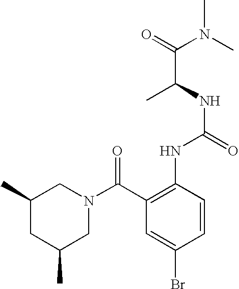 | 453.38 | 453 | (S)-2-{3-[4-Bromo-2-((3R,5S)-3,5-dimethyl-piperidine-1-carbonyl)-phenyl]-ureido}-N,N-dimethyl-propionamide | C |
| I-90 | 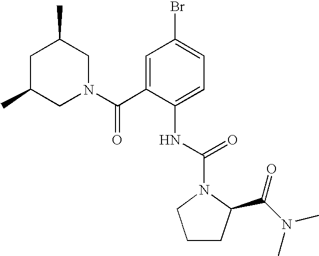 | 479.42 | 479 | (R)-Pyrrolidine-1,2-dicarboxylic acid 1-{[4-bromo-2-((3R,5S)-3,5-dimethyl-piperidine-1-carbonyl)-phenyl]-amide}2-dimethylamide | C |
| I-91 | 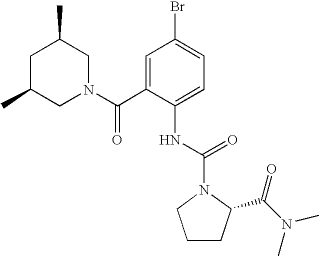 | 479.42 | 479 | (S)-Pyrrolidine-1,2-dicarboxylic acid 1-{[4-bromo-2-((3R,5S)-3,5-dimethyl-piperidine-1-carbonyl)-phenyl]-amide}2-dimethylamide | C |
| I-92 | 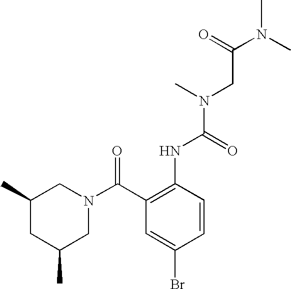 | 453.38 | 453 | 2-{3-[4-Bromo-2-((3R,5S)-3,5-dimethyl-piperidine-1-carbonyl)-phenyl]-1-methyl-ureido}-N,N-dimethyl-acetamide | C |
| I-93 | 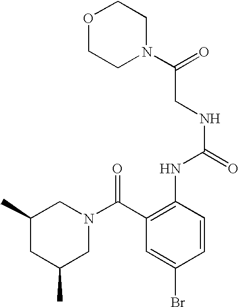 | 481.39 | 481 | 1-[4-Bromo-2-((3S,5R)-3,5-dimethyl-piperidine-1-carbonyl)-phenyl]-3-(2-morpholin-4-yl-2-oxo-ethyl)-urea | C |
| I-94 | 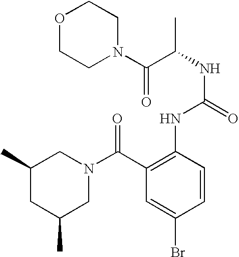 | 495.42 | 495 | 1-[4-Bromo-2-((3S,5R)-3,5-dimethyl-piperidine-1-carbonyl)-phenyl]-3-((S)-1-methyl-2-morpholin-4-yl-2-oxo-ethyl)-urea | C |
| I-95 | 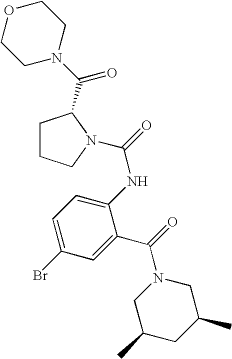 | | 521 | (R)-2-(Morpholine-4-carbonyl)-pyrrolidine-1-carboxylic acid [4-bromo-2-((3R,5S)-3,5-dimethyl-piperidine-1-carbonyl)-phenyl]-amide | |
| I-96 | 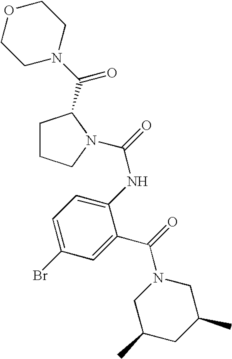 | 521.45 | 521 | (R)-2-(Morpholine-4-carbonyl)-pyrrolidine-1-carboxylic acid [4-bromo-2-((3R,5S)-3,5-dimethyl-piperidine-1-carbonyl)-phenyl]-amide | C |
| I-97 | 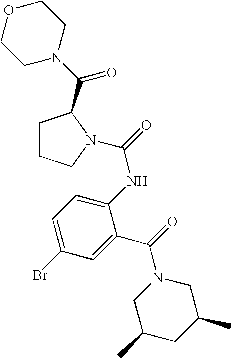 | 521.45 | 521 | (S)-2-(Morpholine-4-carbonyl)-pyrrolidine-1-carboxylic acid [4-bromo-2-((3R,5S)-3,5-dimethyl-piperidine-1-carbonyl)-phenyl]-amide | C |
| I-98 | 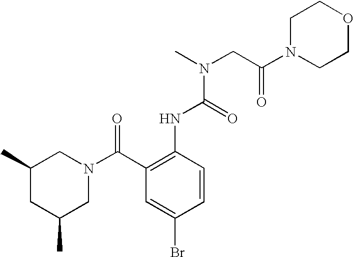 | 495.42 | 495 | 3-[4-Bromo-2-((3S,5R)-3,5-dimethyl-piperidine-1-carbonyl)-phenyl]-1-methyl-1-(2-morpholin-4-yl-2-oxo-ethyl)-urea | C |
| I-99 | 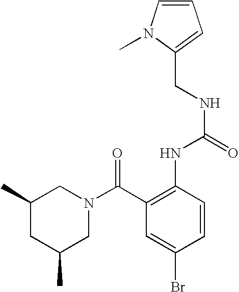 | 447.37 | 447 | 1-[4-Bromo-2-((3S,5R)-3,5-dimethyl-piperidine-1-carbonyl)-phenyl]-3-(1-methyl-1H-pyrrol-2-ylmethyl)-urea | C |
| I-100 | 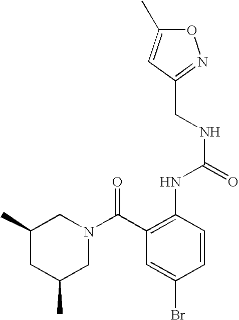 | 449.35 | 449 | 1-[4-Bromo-2-((3R,5S)-3,5-dimethyl-piperidine-1-carbonyl)-phenyl]-3-(5-methyl-isoxazol-3-ylmethyl)-urea | C |
| I-101 | 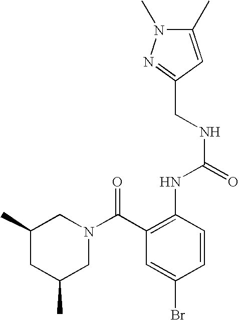 | 462.39 | 462 | 1-[4-Bromo-2-((3R,5S)-3,5-dimethyl-piperidine-1-carbonyl)-phenyl]-3-(1,5-dimethyl-1H-pyrazol-3-ylmethyl)-urea | A |
| I-102 | 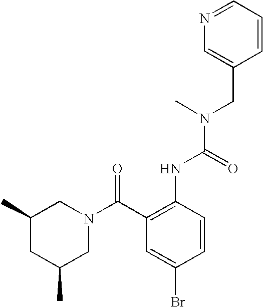 | 459.39 | 459 | 3-[4-Bromo-2-((3R,5S)-3,5-dimethyl-piperidine-1-carbonyl)-phenyl]-1-methyl-1-pyridin-3-ylmethyl-urea | B |
| I-103 | 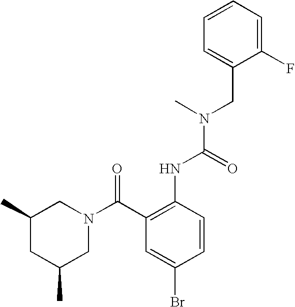 | 476.39 | 476 | 3-[4-Bromo-2-((3R,5S)-3,5-dimethyl-piperidine-1-carbonyl)-phenyl]-1-(2-fluoro-benzyl)-1-methyl-urea | C |
| I-104 | 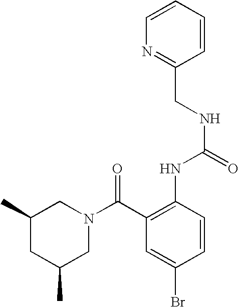 | 445.36 | 445 | 1-[4-Bromo-2-((3S,5R)-3,5-dimethyl-piperidine-1-carbonyl)-phenyl]-3-pyridin-2-ylmethyl-urea | C |
| I-105 | 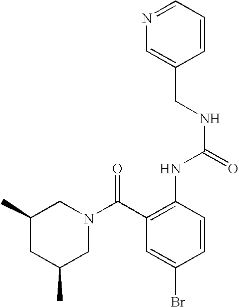 | 445.36 | 445 | 1-[4-Bromo-2-((3S,5R)-3,5-dimethyl-piperidine-1-carbonyl)-phenyl]-3-pyridin-3-ylmethyl-urea | C |
| I-106 | 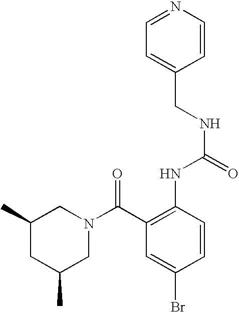 | 445.36 | 445 | 1-[4-Bromo-2-((3S,5R)-3,5-dimethyl-piperidine-1-carbonyl)-phenyl]-3-pyridin-4-ylmethyl-urea | A |
| I-107 | 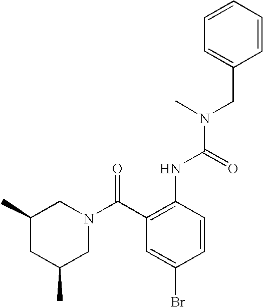 | 458.4 | 458 | 1-Benzyl-3-[4-bromo-2-((3R,5S)-3,5-dimethyl-piperidine-1-carbonyl)-phenyl]-1-methyl-urea | B |
| I-108 | 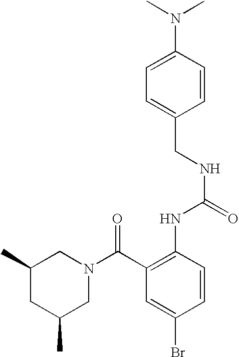 | 487.44 | 487 | 1-[4-Bromo-2-((3S,5R)-3,5-dimethyl-piperidine-1-carbonyl)-phenyl]-3-(4-dimethylamino-benzyl)-urea | B |
| I-109 | 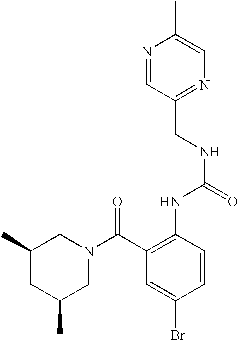 | 460.37 | 460 | 1-[4-Bromo-2-((3R,5S)-3,5-dimethyl-piperidine-1-carbonyl)-phenyl]-3-(5-methyl-pyrazin-2-ylmethyl)-urea | A |
| I-110 | 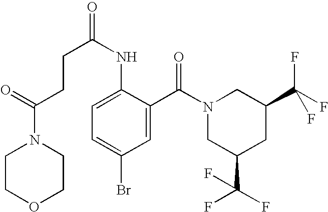 | 588.34 | 588 | N-[2-((3R,5S)-3,5-Bis-trifluoromethyl-piperidine-1-carbonyl)-4-bromo-phenyl]-4-morpholin-4-yl-4-oxo-butyramide | C |
| I-111 | 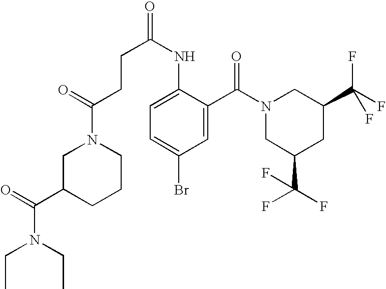 | 685.5 | 685 | 1-{3-[2-((3S,5R)-3,5-Bis-trifluoromethyl-piperidine-1-carbonyl)-4-bromo-phenylcarbamoyl]-propionyl}-piperidine-3-carboxylic aciddiethylamide | B |
| I-112 | 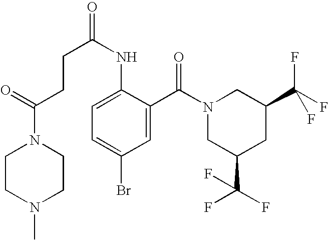 | 601.38 | 601 | N-[2-((3S,5R)-3,5-Bis-trifluoromethyl-piperidine-1-carbonyl)-4-bromo-phenyl]-4-(4-methyl-piperazin-1-yl)-4-oxo-butyramide | C |
| I-113 | 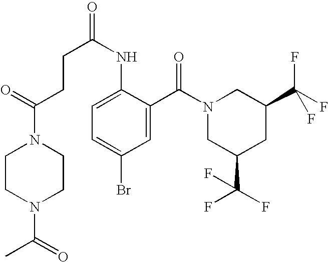 | 629.39 | 629 | 4-(4-Acetyl-piperazin-1-yl)-N-[2-((3R,5S)-3,5-bis-trifluoromethyl-piperidine-1-carbonyl)-4-bromo-phenyl]-4-oxo-butyramide | C |
| I-114 | 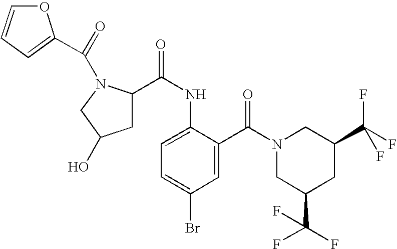 | 626.35 | 626 | 1-(Furan-2-carbonyl)-4-hydroxy-pyrrolidine-2-carboxylic acid [2-((3S,5R)-3,5-bis-trifluoromethyl-piperidine-1-carbonyl)-4-bromo-phenyl]-amide | C |
| I-115 | 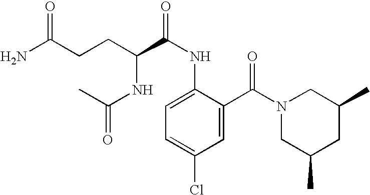 | 436.94 | 437 | (S)-2-Acetylamino-pentanedioic acid 5-amide1-{[4-chloro-2-((3S,5R)-3,5-dimethyl-piperidine-1-carbonyl)-phenyl]-amide} | C |
| I-116 | 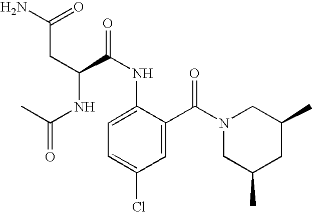 | 422.91 | 423 | (S)-2-Acetylamino-N1-[4-chloro-2-((3S,5R)-3,5-dimethyl-piperidine-1-carbonyl)-phenyl]-succinamide | B |
| I-117 | 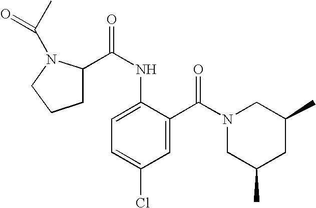 | 405.92 | 406 | 1-Acetyl-pyrrolidine-2-carboxylic acid [4-chloro-2-((3R,5S)-3,5-dimethyl-piperidine-1-carbonyl)-phenyl]-amide | |
| I-118 | 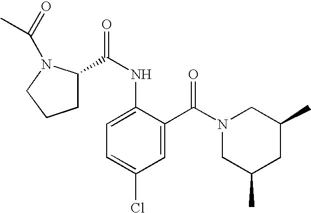 | 405.92 | 406 | (S)-1-Acetyl-pyrrolidine-2-carboxylic acid [4-chloro-2-((3R,5S)-3,5-dimethyl-piperidine-1-carbonyl)-phenyl]-amide | B |
| I-119 | 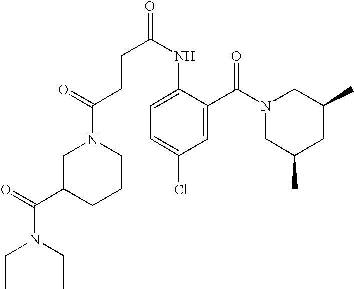 | 533.11 | 533 | 1-{3-[4-Chloro-2-((3S,5R)-3,5-dimethyl-piperidine-1-carbonyl)-phenylcarbamoyl]-propionyl}-piperidine-3-carboxylic aciddiethylamide | A |
| I-120 | 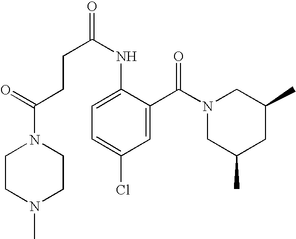 | 448.99 | 449 | N-[4-Chloro-2-((3R,5S)-3,5-dimethyl-piperidine-1-carbonyl)-phenyl]-4-(4-methyl-piperazin-1-yl)-4-oxo-butyramide | C |
| I-121 | 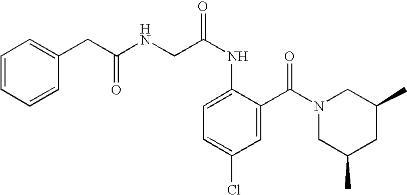 | 441.96 | 442 | N-{[4-Chloro-2-((3R,5S)-3,5-dimethyl-piperidine-1-carbonyl)-phenylcarbamoyl]-methyl}-2-phenyl-acetamide | C |
| I-122 | 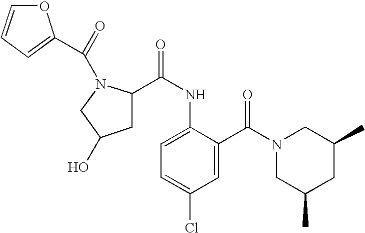 | 473.95 | 474 | 1-(Furan-2-carbonyl)-4-hydroxy-pyrrolidine-2-carboxylic acid [4-chloro-2-((3S,5R)-3,5-dimethyl-piperidine-1-carbonyl)-phenyl]-amide | B |
| I-123 | 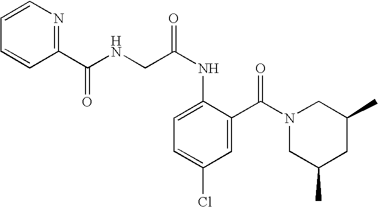 | 428.92 | 429 | Pyridine-2-carboxylic acid{[4-chloro-2-((3S,5R)-3,5-dimethyl-piperidine-1-carbonyl)-phenylcarbamoyl]-methyl}-amide | C |
| I-124 | 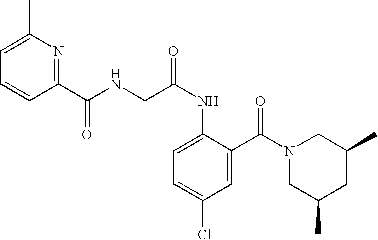 | 442.94 | 443 | 6-Methyl-pyridine-2-carboxylic acid {[4-chloro-2-((3S,5R)-3,5-dimethyl-piperidine-1-carbonyl)-phenylcarbamoyl]-methyl}-amide | C |
| I-125 | 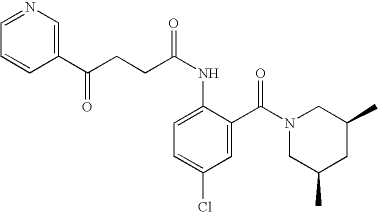 | 427.93 | 428 | N-[4-Chloro-2-((3R,5S)-3,5-dimethyl-piperidine-1-carbonyl)-phenyl]-4-oxo-4-pyridin-2-yl-butyramide | A |
| I-126 | 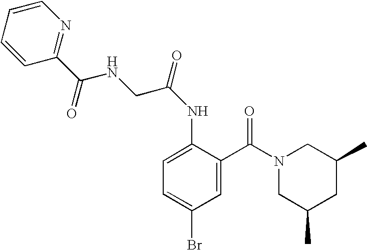 | 473.37 | 473 | Pyridine-2-carboxylic acid{[4-bromo-2-((3S,5R)-3,5-dimethyl-piperidine-1-carbonyl)-phenylcarbamoyl]-methyl}-amide | C |
| I-127 | 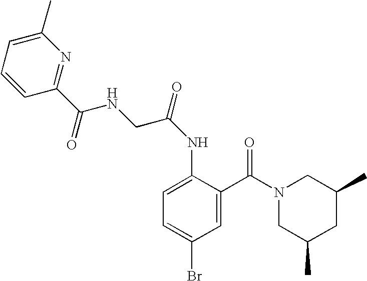 | 487.4 | 487 | 6-Methyl-pyridine-2-carboxylic acid {[4-bromo-2-((3S,5R)-3,5-dimethyl-piperidine-1-carbonyl)-phenylcarbamoyl]-methyl}-amide | C |
| I-128 | 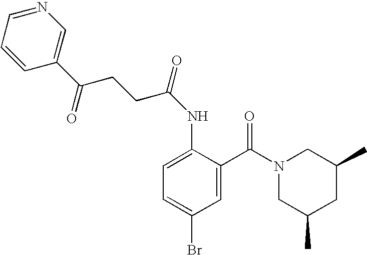 | 472.38 | 472 | N-[4-Bromo-2-((3R,5S)-3,5-dimethyl-piperidine-1-carbonyl)-phenyl]-4-oxo-4-pyridin-3-yl-butyramide | B |
| I-129 | 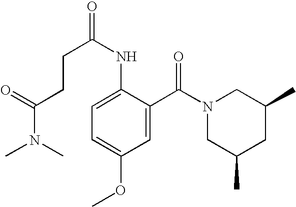 | 389.49 | 390 | N-[2-((3S,5R)-3,5-Dimethyl-piperidine-1-carbonyl)-4-methoxy-phenyl]-N′,N′-dimethyl-succinamide | A |
| I-130 | 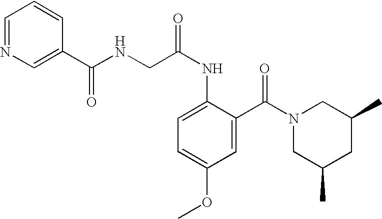 | 424.5 | 425 | N-{[2-((3R,5S)-3,5-Dimethyl-piperidine-1-carbonyl)-4-methoxy-phenylcarbamoyl]-methyl}-nicotinamide | A |
| I-131 | 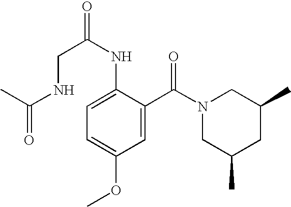 | 361.44 | 362 | 2-Acetylamino-N-[2-((3R,5S)-3,5-dimethyl-pipeidine-1-carbonyl)-4-methoxy-phenyl]-acetamide | A |
| I-132 | 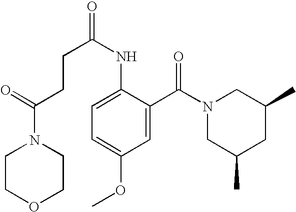 | 431.53 | 432 | N-[2-((3R,5S)-3,5-Dimethyl-piperidine-1-carbonyl)-4-methoxy-phenyl]-4-morpholin-4-yl-4-oxo-butyramide | A |
| I-133 | 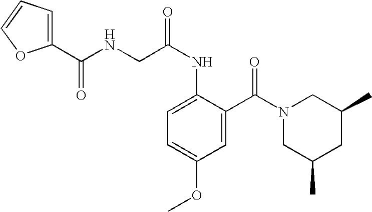 | 413.47 | 414 | Furan-2-carboxylic acid{[2-((3R,5S)-3,5-dimethyl-piperidine-1-carbonyl)-4-methoxy-phenylcarbamoyl]-methyl}-amide | A |
| I-134 | 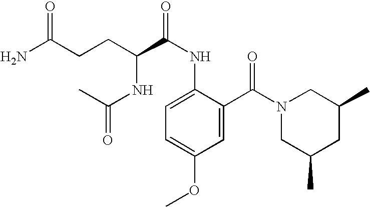 | 432.52 | 433 | (S)-2-Acetylamino-pentanedioic acid 5-amide1-{[2-((3R,5S)-3,5-dimethyl-piperidine-1-carbonyl)-4-methoxy-phenyl]-amide} | A |
| I-135 | 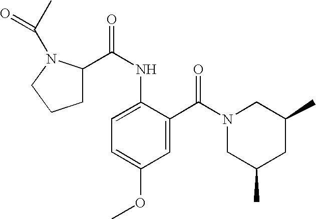 | 401.5 | 402 | 1-Acetyl-pyrrolidine-2-carboxylic acid [2-((3S,5R)-3,5-dimethyl-piperidine-1-carbonyl)-4-methoxy-phenyl]-amide | A |
| I-136 | 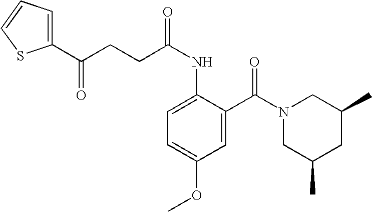 | 428.55 | 429 | N-[2-((3R,5S)-3,5-Dimethyl-piperidine-1-carbonyl)-4-methoxy-phenyl]-4-oxo-4-thiophen-2-yl-butyramide | C |
| I-137 | 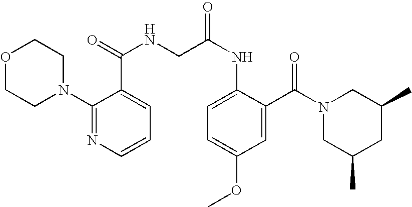 | 509.6 | 510 | N-{[2-((3R,5S)-3,5-Dimethyl-piperidine-1-carbonyl)-4-methoxy-phenylcarbamoyl]-methyl}-2-morpholin-4-yl-nicotinamide | C |
| I-138 | 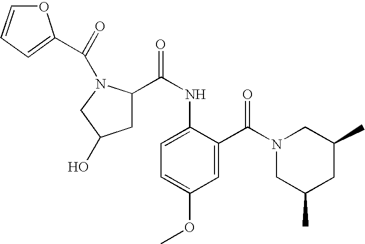 | 469.54 | 470 | 1-(Furan-2-carbonyl)-4-hydroxy-pyrrolidine-2-carboxylic acid [2-((3R,5S)-3,5-dimethyl-piperidine-1-carbonyl)-4-methoxy-phenyl]-amide | C |
| I-139 | 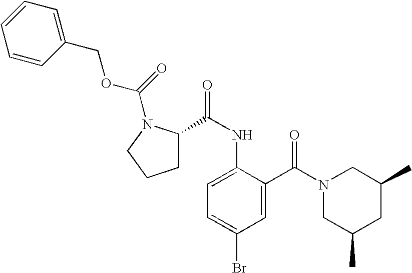 | 542.47 | 542 | (S)-2-[4-Bromo-2-((3S,5R)-3,5-dimethyl-piperidine-1-carbonyl)-phenylcarbamoyl]-pyrrolidine-1-carboxylicacid benzyl ester | C |
| I-140 | 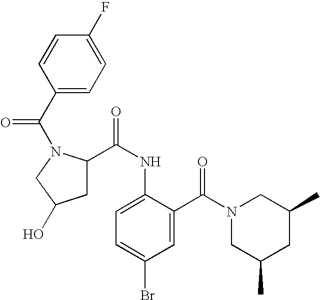 | 546.43 | 546 | 1-(4-Fluoro-benzoyl)-4-hydroxy-pyrrolidine-2-carboxylic acid [4-bromo-2-((3S,5R)-3,5-dimethyl-piperidine-1-carbonyl)-phenyl]-amide | B |
| I-141 | 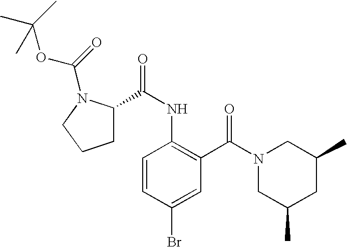 | 508.45 | 508 | (S)-2-[4-Bromo-2-((3S,5R)-3,5-dimethyl-piperidine-1-carbonyl)-phenylcarbamoyl]-pyrrolidine-1-carboxylicacid tert-butyl ester | C |
| I-142 | 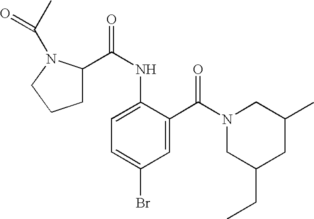 | 464.4 | 464 | 1-Acetyl-pyrrolidine-2-carboxylic acid [4-bromo-2-(3-ethyl-5-methyl-piperidine-1-carbonyl)-phenyl]-amide | C |
| I-143 | 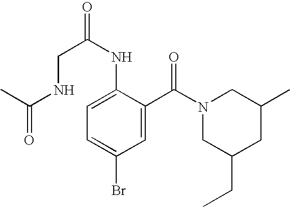 | 424.34 | 424 | 2-Acetylamino-N-[4-bromo-2-(3-ethyl-5-methyl-piperidine-1-carbonyl)-phenyl]-acetamide | B |
| I-144 | 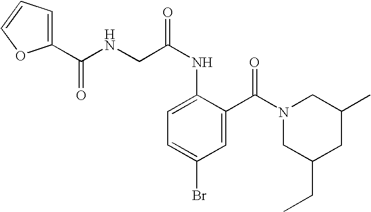 | 476.37 | 476 | Furan-2-carboxylic acid{[4-bromo-2-(3-ethyl-5-methyl-piperidine-1-carbonyl)-phenylcarbamoyl]-methyl}-amide | B |
| I-145 | 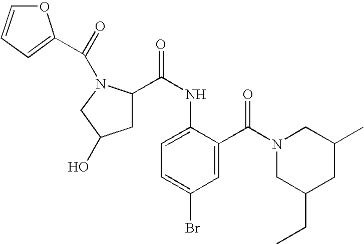 | 532.43 | 532 | 1-(Furan-2-carbonyl)-4-hydroxy-pyrrolidine-2-carboxylic acid [4-bromo-2-(3-ethyl-5-methyl-piperidine-1-carbonyl)-phenyl]-amide | B |
| I-146 | 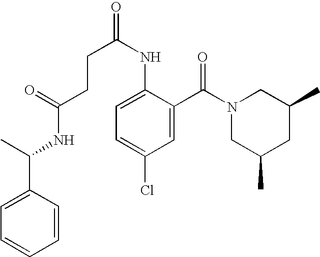 | 470.01 | 470 | N-[4-Chloro-2-((3S,5R)-3,5-dimethyl-piperidine-1-carbonyl)-phenyl]-N′-((S)-1-phenyl-ethyl)-succinamide | C |
| I-147 | 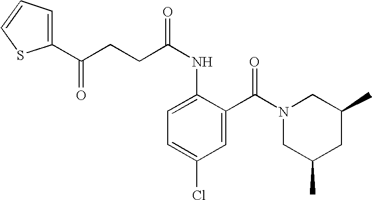 | 432.97 | 433 | N-[4-Chloro-2-((3R,5S)-3,5-dimethyl-piperidine-1-carbonyl)-phenyl]-4-oxo-4-thiophen-2-yl-butyramide | C |
| I-148 | 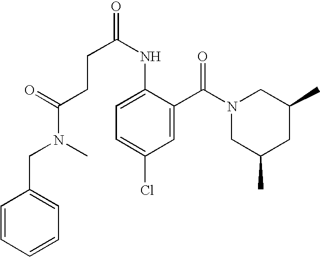 | 470.01 | 470 | N-Benzyl-N′-[4-chloro-2-((3R,5S)-3,5-dimethyl-piperidine-1-carbonyl)-phenyl]-N-methyl-succinamide | A |
| I-149 | 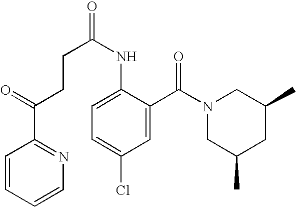 | 427.93 | 428 | N-[4-Chloro-2-((3R,5S)-3,5-dimethyl-piperidine-1-carbonyl)-phenyl]-4-oxo-4-pyridin-2-yl-butyramide | C |
| I-150 | 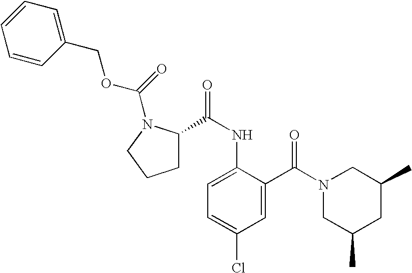 | 498.02 | 498 | (S)-2-[4-Chloro-2-((3S,5R)-3,5-dimethyl-piperidine-1-carbonyl)-phenylcarbamoyl]-pyrrolidine-1-carboxylicacid benzyl ester | C |
| I-151 | 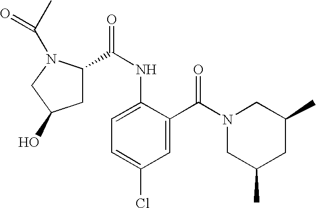 | 421.92 | 422 | (2S,4R)-1-Acetyl-4-hydroxy-pyrrolidine-2-carboxylic acid [4-chloro-2-((3S,5R)-3,5-dimethyl-piperidine-1-carbonyl)-phenyl]-amide | C |
| I-152 | 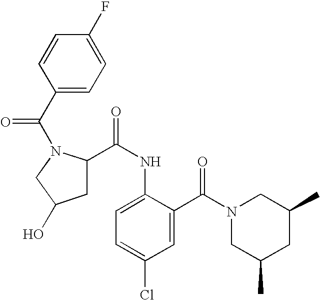 | 501.98 | 502 | 1-(4-Fluoro-benzoyl)-4-hydroxy-pyrrolidine-2-carboxylic acid [4-chloro-2-((3S,5R)-3,5-dimethyl-piperidine-1-carbonyl)-phenyl]-amide | C |
| I-153 | 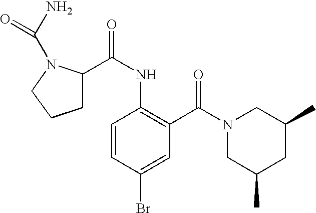 | 451.36 | 451 | Pyrrolidine-1,2-dicarboxylic acid 1-amide2-{[4-bromo-2-((3S,5R)-3,5-dimethyl-piperidine-1-carbonyl)-phenyl]-amide} | C |
| I-154 | 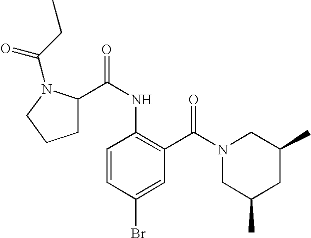 | 464.4 | 464 | 1-Propionyl-pyrrolidine-2-carboxylic acid [4-bromo-2-((3S,5R)-3,5-dimethyl-piperidine-1-carbonyl)-phenyl]-amide | B |
| I-155 | 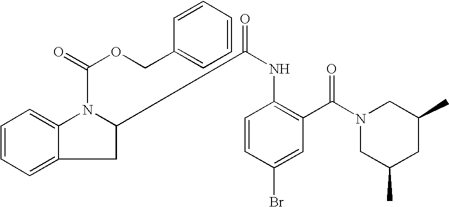 | 590.52 | 590 | 2-[4-Bromo-2-((3R,5S)-3,5-dimethyl-piperidine-1-carbonyl)-phenylcarbamoyl]-2,3-dihydro-indole-1-carboxylicacid benzyl ester | C |
| I-156 | 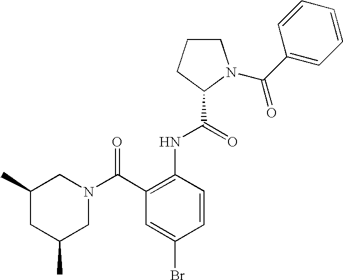 | 512.45 | 512 | (S)-1-Benzoyl-pyrrolidine-2-carboxylic acid [4-bromo-2-((3S,5R)-3,5-dimethyl-piperidine-1-carbonyl)-phenyl]-amide | C |
| I-157 | 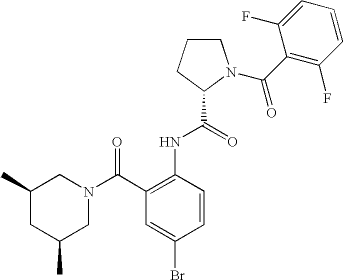 | 548.43 | 548 | (S)-1-(2,6-Difluoro-benzoyl)-pyrrolidine-2-carboxylic acid [4-bromo-2-((3S,5R)-3,5-dimethyl-piperidine-1-carbonyl)-phenyl]-amide | C |
| I-158 | 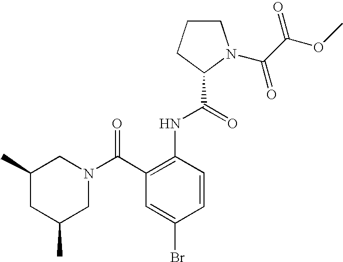 | 494.38 | 494 | {(S)-2-[4-Bromo-2-((3S,5R)-3,5-dimethyl-piperidine-1-carbonyl)-phenylcarbamoyl]-pyrrolidin-1-yl}-oxo-aceticacid methyl ester | C |
| I-159 | 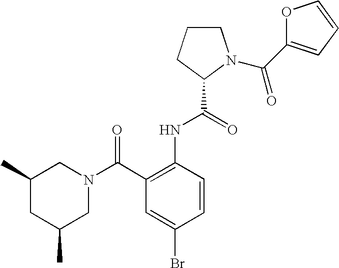 | 502.41 | 502 | (S)-1-(Furan-2-carbonyl)-pyrrolidinyl-2-carboxylicacid [4-bromo-2-((3S,5R)-3,5-dimethyl-piperidine-1-carbonyl)-phenyl]-amide | A |
| I-160 | 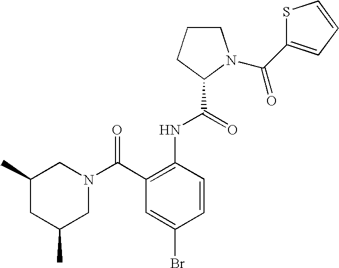 | 518.47 | 518 | (S)-1-(Thiophene-2-carbonyl)-pyrrolidine-2-carboxylicacid [4-bromo-2-((3S,5R)-3,5-dimethyl-piperidine-1-carbonyl)-phenyl]-amide | A |
| I-161 | 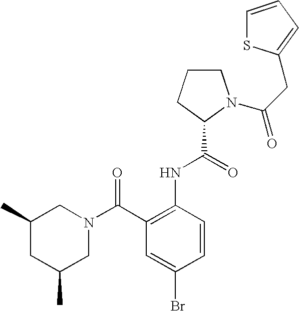 | 532.5 | 532 | (S)-1-(2-Thiophen-2-yl-acetyl)-pyrrolidine-2-carboxylicacid [4-bromo-2-((3S,5R)-3,5-dimethyl-piperidine-1-carbonyl)-phenyl]-amide | C |
| I-162 | 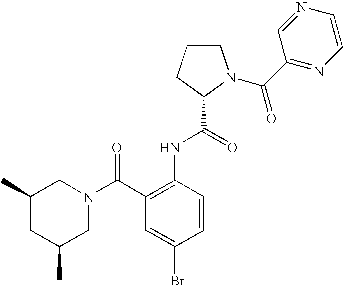 | 514.42 | 514 | (S)-1-(Pyrazine-2-carbonyl)-pyrrolidine-2-carboxylicacid [4-bromo-2-((3S,5R)-3,5-dimethyl-piperidine-1-carbonyl)-phenyl]-amide | C |
| I-163 | 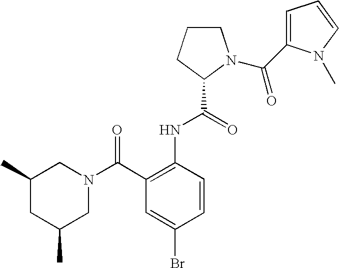 | 515.45 | 515 | (S)-1-(1-Methyl-1H-pyrrole-2-carbonyl)-pyrrolidine-2-carboxylicacid [4-bromo-2-((3S,5R)-3,5-dimethyl-piperidine-1-carbonyl)-phenyl]-amide | C |
| I-164 | 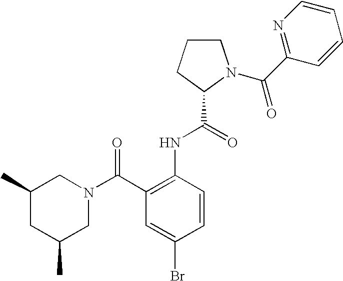 | 513.43 | 513 | (S)-1-(Pyridine-2-carbonyl)-pyrrolidine-2-carboxylic acid [4-bromo-2-((3S,5R)-3,5-dimethyl-piperidine-1-carbonyl)-phenyl]-amide | C |
| I-165 | 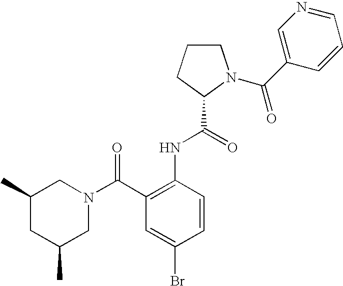 | 513.43 | 513 | (S)-1-(Pyridine-3-carbonyl)-pyrrolidine-2-carboxylic acid [4-bromo-2-((3S,5R)-3,5-dimethyl-piperidine-1-carbonyl)-phenyl]-amide | A |
| I-166 | 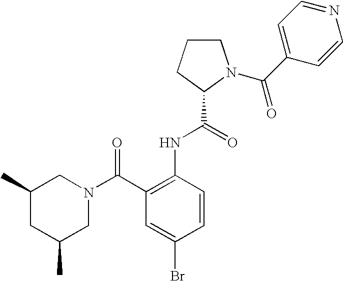 | 513.43 | 513 | (S)-1-(Pyridine-4-carbonyl)-pyrrolidine-2-carboxylic acid [4-bromo-2-((3S,5R)-3,5-dimethyl-piperidine-1-carbonyl)-phenyl]-amide | C |
| I-167 | 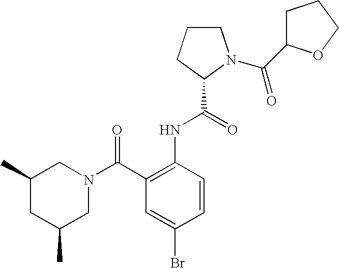 | 506.44 | 506 | (S)-1-(Tetrahydro-furan-2-carbonyl)-pyrrolidine-2-carboxylic acid [4-bromo-2-((3S,5R)-3,5-dimethyl-piperidine-1-carbonyl)-phenyl]-amide | B |
| I-168 | 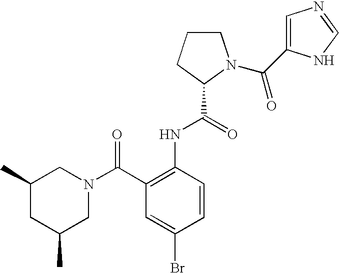 | 502.41 | 502 | (S)-1-(3H-Imidazole-4-carbonyl)-pyrrolidine-2-carboxylic acid [4-bromo-2-((3S,5R)-3,5-dimethyl-piperidine-1-carbonyl)-phenyl]-amide | A |
| I-169 | 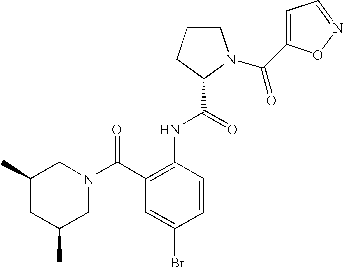 | 503.39 | 503 | (S)-1-(Isoxazole-5-carbonyl)-pyrrolidine-2-carboxylic acid [4-bromo-2-((3S,5R)-3,5-dimethyl-piperidine-1-carbonyl)-phenyl]-amide | A |
| I-170 | 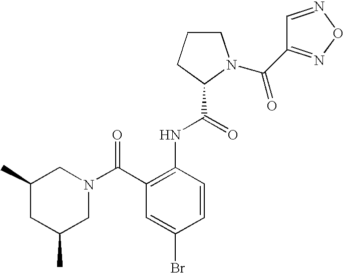 | 504.38 | 504 | (S)-1-(Furazan-3-carbonyl)-pyrrolidine-2-carboxylic acid [4-bromo-2-((3S,5R)-3,5-dimethyl-piperidine-1-carbonyl)-phenyl]-amide | C |
| I-171 | 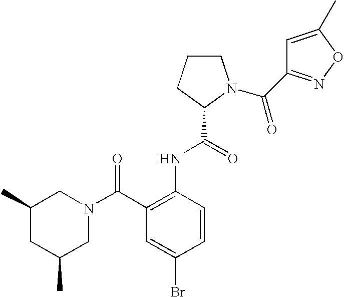 | 517.42 | 517 | (S)-1-(5-Methyl-isoxazole-3-carbonyl)-pyrrolidine-2-carboxylic acid [4-bromo-2-((3S,5R)-3,5-dimethyl-piperidine-1-carbonyl)-phenyl]-amide | B |
| I-172 | 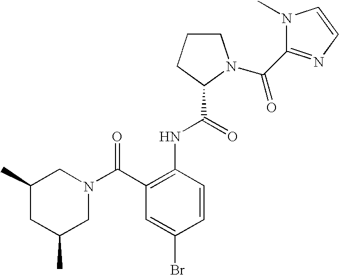 | 516.44 | 516 | (S)-1-(1-Methyl-1H-imdazole-2-carbonyl)-pyrrolidine-2-carboxylicacid [4-bromo-2-((3S,5R)-3,5-dimethyl-piperidine-1-carbonyl)-phenyl]-amide | C |
| I-173 | 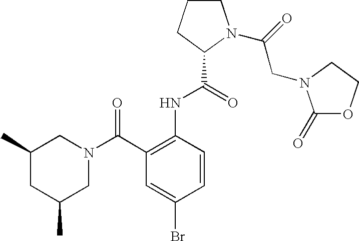 | 535.44 | 535 | (S)-1-[2-(2-Oxo-oxazolidin-3-yl)-acetyl]-pyrrolidine-2-carboxylic acid [4-bromo-2-((3S,5R)-3,5-dimethyl-piperidine-1-carbonyl)-phenyl]-amide | B |
| I-174 | 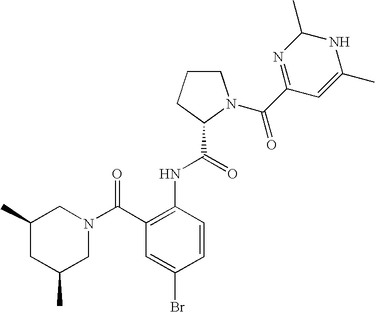 | 544.45 | 544 | (S)-1-(6-Methyl-2-oxo-1,2-dihydro-pyrimidine-4-carbonyl)-pyrrolidine-2-carboxylic acid [4-bromo-2-((3S,5R)-3,5-dimethyl-piperidine-1-carbonyl)-phenyl]-amide | B |
| I-175 | 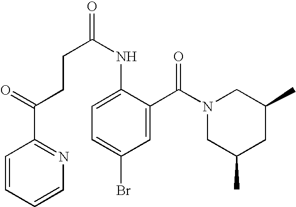 | 472.38 | 472 | N-[4-Bromo-2-((3R,5S)-3,5-dimethyl-piperidine-1-carbonyl)-phenyl]-4-oxo-4-pyridin-2-yl-butyramide | A |
| I-176 | 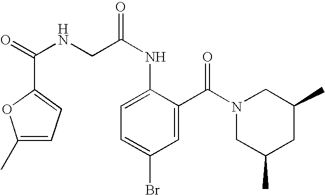 | 476.37 | 476 | 5-Methyl-furan-2-carboxylic acid {[4-bromo-2-((3S,5R)-3,5-dimethyl-piperidine-1-carbonyl)-phenylcarbamoyl]-methyl}-amide | C |
| I-177 | 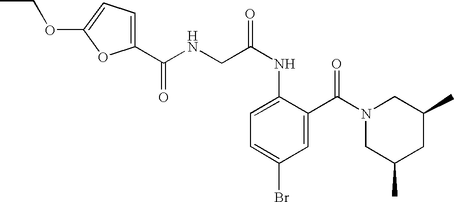 | 506.39 | 506 | 5-Ethoxy-furan-2-carboxylic acid {[4-bromo-2-((3R,5S)-3,5-dimethyl-piperidine-1-carbonyl)-phenylcarbamoyl]-methyl}-amide | C |
| I-178 | 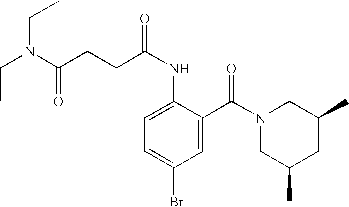 | 466.42 | 466 | N-[4-Bromo-2-((3S,5R)-3,5-dimethyl-piperidine-1-carbonyl)-phenyl]-N′,N′-diethyl-succinamide | C |
| I-179 | 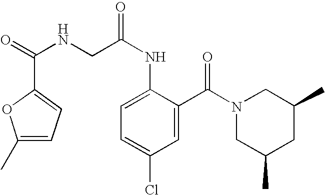 | 431.92 | 432 | 5-Methyl-furan-2-carboxylic acid {[4-chloro-2-((3S,5R)-3,5-dimethyl-piperidine-1-carbonyl)-phenylcarbamoyl]-methyl}-amide | C |
| I-180 | 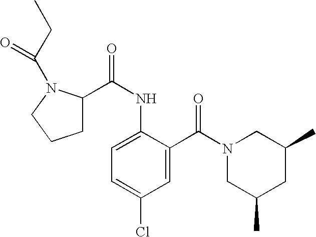 | 419.95 | 420 | 1-Propionyl-pyrrolidine-2-carboxylic acid [4-chloro-2-((3S,5R)-3,5-dimethyl-piperidine-1-carbonyl)-phenyl]-amide | C |
| I-181 | 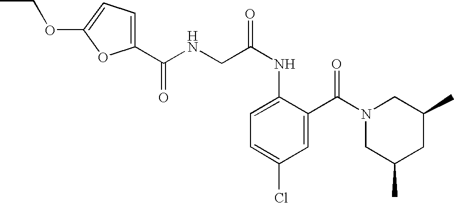 | 461.94 | 462 | 5-Ethoxy-furan-2-carboxylic acid {[4-chloro-2-((3R,5S)-3,5-dimethyl-piperidine-1-carbonyl)-phenylcarbamoyl]-methyl}-amide | C |
| I-182 | 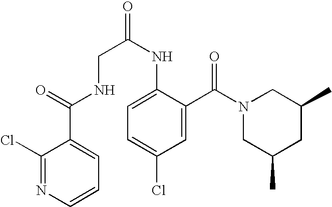 | 463.36 | 463 | 2-Chloro-N-{[4-chloro-2-((3S,5R)-3,5-dimethyl-piperidine-1-carbonyl)-phenylcarbamoyl]-methyl}-nicotinamide | C |
| I-183 | 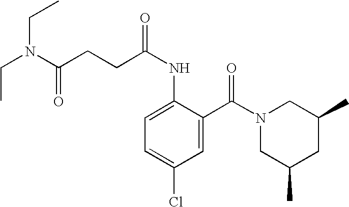 | 421.97 | 422 | N-[4-Chloro-2-((3S,5R)-3,5-dimethyl-piperidine-1-carbonyl)-phenyl]-N′,N′-diethyl-succinamide | B |
| I-184 | 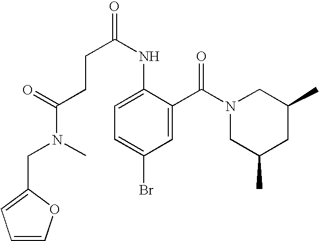 | 504.42 | 504 | N-[4-Bromo-2-((3S,5R)-3,5-dimethyl-piperidine-1-carbonyl)-phenyl]-N′-furan-2-ylmethyl-N′-methyl-succinamide | B |
| I-185 | 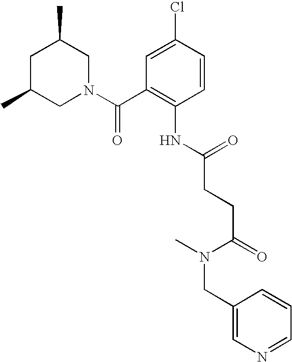 | 471 | 471 | N-[4-Chloro-2-((3R,5S)-3,5-dimethyl-piperidine-1-carbonyl)-phenyl]-N′-methyl-N′-pyridin-3-ylmethyl-succinamide | A |
| I-186 | 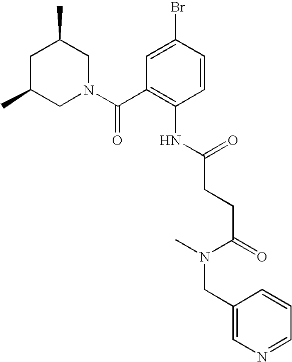 | 515.45 | 515 | N-[4-Bromo-2-((3R,5S)-3,5-dimethyl-piperidine-1-carbonyl)-phenyl]-N′-methyl-N′-pyridin-3-ylmethyl-succinamide | B |

¹A = <0.5 mM, B = 0.5-1.0 mM, C = >1.0 mM HCV polymerase assay

Other aspects of the invention provide a method for treating hepatitis C viral infection in a subject by administering to a subject in need of such treatment a compound of formula I.

Still other aspects of the invention provide methods for making compounds of formula I and pharmaceutical compositions comprising a compound of formula I.

### Synthesis

Compounds of the invention can be made by a variety of methods depicted in the illustrative examples shown in the Examples section below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. The following synthetic reaction schemes are merely illustrative of some methods by which the compounds of the invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reaction described herein preferably are conducted under inert atmosphere, at atmospheric pressure, at a reaction temperature range of from about −78° C. to about 230° C., and most preferably and conveniently at room (or ambient) temperature, e.g., about 20° C.

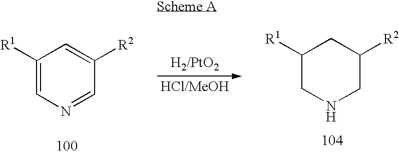

Scheme A illustrates a method for producing optionally substituted piperidine compounds. Such piperidine compounds are then used as the piperidine portion of a compound of Formula I. As shown in Scheme A, reduction of an optionally substituted pyridine compound 100 with platinum oxide in hydrogen atmosphere produces a piperidine compound 104. Typically, the hydrogenation reaction is conducted in the presence of an acid in an alcoholic solvent. Other suitable reduction conditions known to one skilled in the art can also be used. By starting with appropriately substituted pyridine compound 100, one can obtain piperidine compound 104 with a desired substituent(s) and/or substitution patterns. In some instances, the substituent(s) of piperidine compound 104 can be further transformed to yield other desired piperidine compounds. Suitable substituent transformation reactions are well known to one skilled in the art. In some cases one skilled in the art will appreciate that protection of one or more of the functional groups may be necessary in order to provide a selected transformation of a desired functional group. In Scheme A, each of R¹ and R² is independently a variety of substituents, for example, hydrogen, alkyl, haloalkyl, halide, cyano, nitro, protected amino group (including mono and dialkyl amino groups), carboxylic acid and derivatives thereof, etc. It should be appreciated that some substituents, such as nitro and cyano groups can themselves undergo reduction to produce amino and aminoalkyl groups, respectively. Typically, each of R¹ and R² is independently hydrogen, alkyl, or haloalkyl.

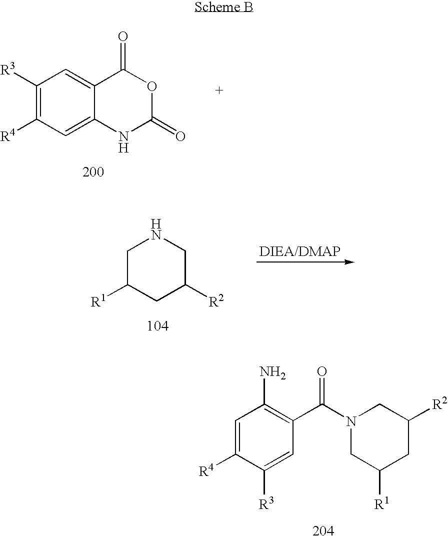

Scheme B illustrates one method of forming the arylpiperidine ketone compound 204 that is used in preparing Compounds of Formula I. As shown in Scheme B, an isatoic anhydride 200 is reacted with a piperidine compound 104 in the presence of a base, e.g., diisopropylethylamine (DIEA), and an acyl transfer catalyst, e.g., dimethylaminopyridine (DMAP). Typically, this reaction is conducted in DMF at room temperature. Other acyl transfer reactions and/or reagents can also be used. In Scheme B, R¹-R⁴ are those defined herein.

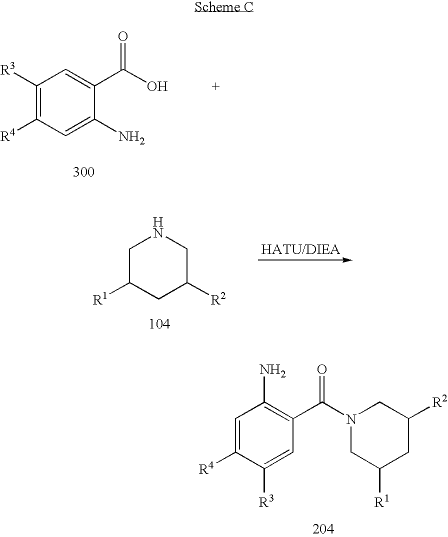

Scheme C illustrates another method for forming the arylpiperidine ketone compound 204. In this method a carboxylic acid 300 is coupled with a piperidine compound 104 using a coupling reagent O-(7-azabenzotriazole-1-yl)-N,N,N′N′-tetramethyluronium hexafluorophosphate (HATU) in the presence of a base, e.g., DIEA. Typically this reaction is conducted at an elevated temperature, e.g., 90° C., in DMF. Other coupling reagents and/or bases can also be used.

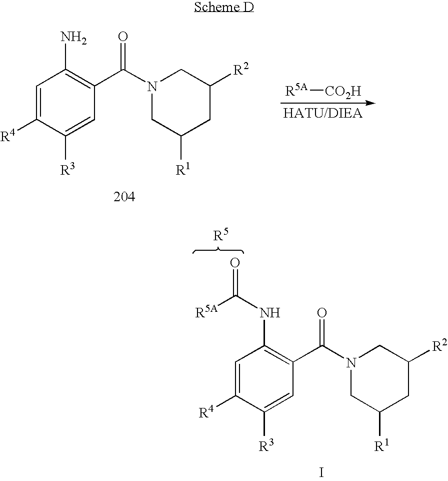

Scheme D illustrates one of the methods for coupling the amino group of the aryl moiety with an amino acid, succinic acid, or a derivative thereof. As illustrated in Scheme D, the R^{5A}—CO₂— moiety corresponds to the R⁵ moiety of Compound of Formula I. As can be seen in Scheme D, the reaction utilizes coupling reaction between the amino group and a carboxylic acid group. Any suitable standard coupling reaction conditions can be used, such as HATU/DIEA combination as illustrated in Scheme D.

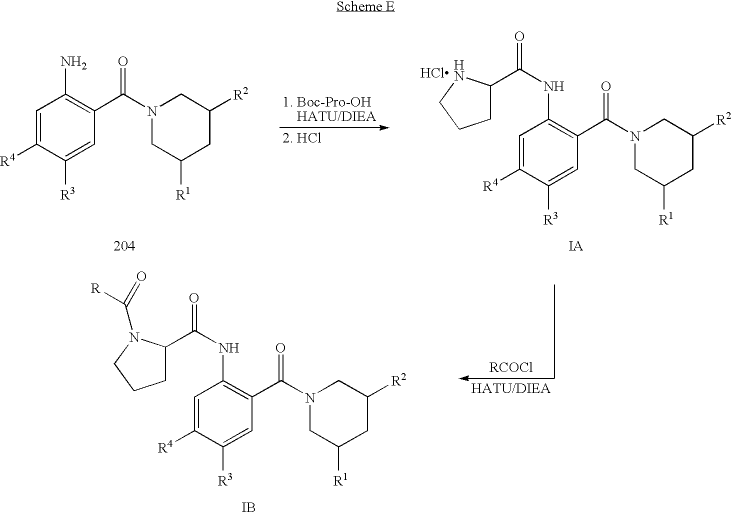

Another method for coupling an amino acid with an arylpiperidine ketone compound 204 is shown in Scheme E. In this method, the carboxylic acid group of an amino acid with its amino group protected (e.g., Boc-Pro-OH) is coupled with the amino group of an arylpiperidine ketone compound 204 using a typical amide functional group formation reaction followed by removal of the Boc protecting group yields a compound of Formula IA. As shown in Scheme E, the free amino group of the amino acid moiety can be further transformed to compound IB, for example, to an amide group by reacting with an acyl chloride.

One of the methods for synthesizing compounds of Formula I with a urea functional group is illustrated in Scheme F below. In this method, the amino group in the aromatic ring is first converted to an isocyanate group by reacting an arylpiperidine ketone compound 204 with phosgene in the presence of a mild base such as a bicarbonate, e.g., sodium bicarbonate. Typically an excess amount of phosgene is used in this reaction to ensure a high yield of the isocyanate intermediate 600. Reacting the isocyanate intermediate 600 with a compound comprising an amine functional group, e.g., an amino acid or an amino compound, results in the urea compound of Formula IC. In this manner, a wide variety of urea derivatives of Compounds of Formula I can be prepared.

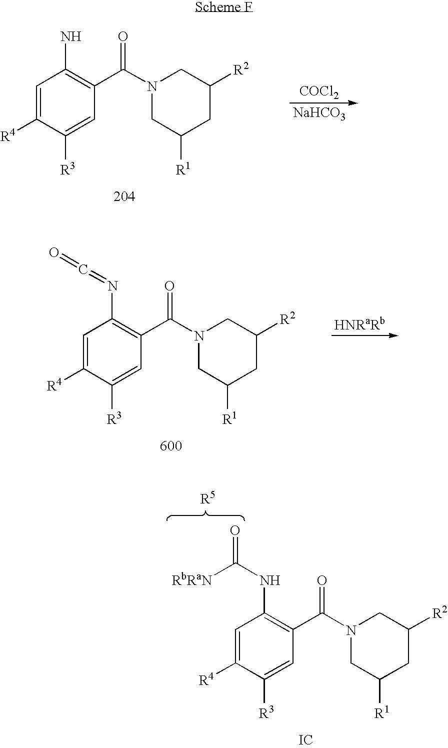

### Administration and Pharmaceutical Composition

The invention includes pharmaceutical compositions comprising at least one compound of the present invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients.

In general, the compounds of the invention is administered in a therapeutically effective amount by any of the accepted modes of administration for agents that serve similar utilities. Suitable dosage ranges are typically 1-500 mg daily, preferably 1-100 mg daily, and most preferably 1-30 mg daily, depending upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compounds of the present invention for a given disease.

Compounds of the invention can be administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation. The preferred manner of administration is generally oral using a convenient daily dosage regimen which can be adjusted according to the degree of affliction.

A compound or compounds of the invention, together with one or more conventional adjuvants, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

The compounds of the invention may be formulated in a wide variety of oral administration dosage forms. The pharmaceutical compositions and dosage forms may comprise a compound or compounds of the present invention or pharmaceutically acceptable salts thereof as the active component. The pharmaceutically acceptable carriers may be either solid or liquid. Solid form preparations include powders, tablets, pills, capsules, cachets, suppositories, and dispersible granules. A solid carrier may be one or more substances which may also act as diluents, flavouring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The compounds of the invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of the invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

The compounds of the invention can be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the invention can be formulated for vaginal administration. Pessaries, tampons, creams, gels, pastes, foams or sprays containing in addition to the active ingredient such carriers as are known in the art to be appropriate.

The subject compounds can be formulated for nasal administration. The solutions or suspensions are applied directly to the nasal cavity by conventional means, for example, with a dropper, pipette or spray. The formulations can be provided in a single or multidose form. In the latter case of a dropper or pipette, this can be achieved by the patient administering an appropriate, predetermined volume of the solution or suspension. In the case of a spray, this can be achieved for example by means of a metering atomizing spray pump.

The compounds of the invention can be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatine or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylazacycloheptan-2-one). Sustained release delivery systems are inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa. Representative pharmaceutical formulations containing a compound of the present invention are described below.

Additional objects, advantages, and novel features of this invention will become apparent to those skilled in the art upon examination of the following examples thereof, which are not intended to be limiting.

### EXAMPLES

### General Procedures

HPLC-MS: Unless otherwise indicated final products were purified by mass-triggered reverse phase HPLC on a Waters Autopurification system. The stationary phase was a 50×21 mm ODS-18 5 um column, the mobile phase was a gradient of 5 to 95% Buffer B over 10 minutes (Buffer A=95% H₂O, 5% ACN containing 0.1% formic acid; Buffer B=100% ACN containing 0.1% formic acid). Fractions containing the desired product were concentrated and then dissolved to 10 mM in DMSO for screening. Final product purity and identity was verified by analytical HPLC-MS.

### Example 1

This example illustrates synthesis of 3,5-bis-trifluoromethylpiperidine.

To a solution of 3,5-bis-trifluoromethylpyridine (1.14 g. 5.3 mmol) in 100 ml of methanol was added 5.3 mmol (0.44 ml) of 12N HCl and 100 mg of PtO₂ (Adam's catalyst) under N₂. The solution was then placed on a Parr shaker, purged and shaken under 60 PSI of H₂ gas for 48 hours. The resulting mixture was diluted with 5 ml of water and the catalyst was filtered on a bed of Celite. The solvents were removed and the product co-evaporated with acetonitrile (ACN) to yield 1.86 g of white solid product (95%). ¹H NMR in DMSO-d₆ showed no aromatic protons indicating reduction of the pyridine.

### Example 2

This example illustrates synthesis of 3-ethylpiperidine.

This material was prepared according to the procedure described in Example 1 using 3-ethylpyridine as the starting material.

### Example 3

This example illustrates synthesis of 3-methyl-5-ethylpiperidine.

This material was prepared according to the procedure described in Example 1 using 3-methyl-5-ethylpyridine as the starting material.

### Example 4

This example illustrates synthesis of 3-isobutylpiperidine.

This material was prepared according to the procedure described in Example 1 using 3-methyl-5-ethylpyridine as the starting material.

### Example 5

This example illustrates synthesis of 2-aminoaryl piperidine carboxamides from isatoic anhydrides.

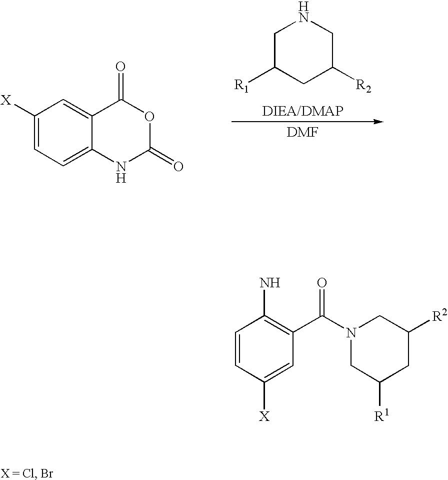

The isatoic anhydride (2.1 mmol, 1.2 equiv, 5-bromoisatoic anhydride or 5-chloroisatoic anhydride), 3,5-dimethyl piperidine or the piperidines from examples 1 to 4 (1.7 mmol, 1 equiv), DMAP (1.7 mmol, 0.1 equiv), and DIEA (2.1 mmol, 1.2 equiv) were dissolved in DMF (66 mL). The reaction mixture was stirred at room temperature (RT) overnight. DMF was then removed in vacuo and the residue purified by Isco CombiFlash Companion (SiO₂, gradient 0-50% ethyl acetate in hexanes) to afford the title compounds in 77-85% yields. The structures were confirmed by LC-MS and ¹H NMR spectroscopy.

### Example 6

This example illustrates synthesis of (5-substituted-2-amino-phenyl)((3R,5S)-3,5-dimethylpiperidin-1-yl)methanones, 2-aminoaryl piperidine carboxamides from 2-aminobenzoic acids.

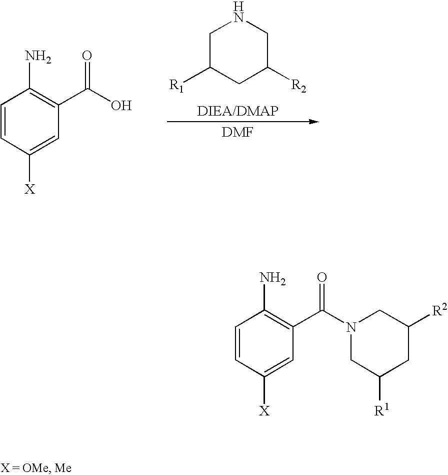

The 2-amino benzoic acid (for example 2-amino-5-methoxybenzoic acid or 2-amino-5-methylbenzoic acid) (6.62 mmol, 1 equiv), 3,5-dimethyl piperidine or the piperidines from examples 1 to 4 (7.95 mmol, 1.2 equiv), HATU (7.95 mmol, 1.2 equiv), and DIEA (7.95 mmol, 1.2 equiv) were dissolved in DMF (29 mL) and stirred overnight at 90° C. The DMF was removed and the residue purified by the Isco CombiFlash Companion system (SiO₂, gradient 1-50% ethyl acetate in hexanes) to provide quantitative yield of the title compounds. The structures were confirmed by LC-MS and ¹H NMR spectroscopy.

### Example 7

This example illustrates synthesis of (5-bromo-2-isocyanato-phenyl)-(3,5-dimethyl-piperidin-1-yl)-methanone.

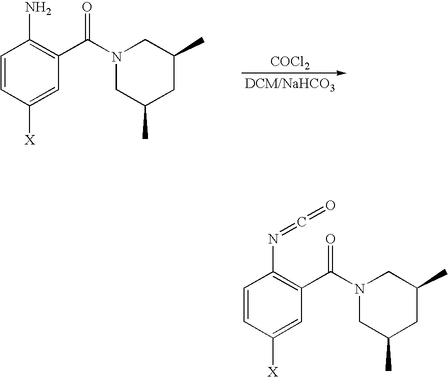

To a 0° C. solution of (5-bromo-2-aminophenyl)-(3,5-dimethyl-piperidin-1-yl)-methanone (0.622 g, 2.0 mmol, 1 equiv) in dichloromethane (DCM) (25 mL) was added a saturated aqueous solution of NaHCO₃. The resulting solution was stirred for an additional 10 min. Phosgene (5.3 mL, 20% solution in toluene, 10 mmol, 5 equiv) was then added and the solution stirred vigorously for 2 h. The aqueous and organic layers were separated and the aqueous layer extracted with DCM (2×). The organic layers were combined and dried over MgSO₄ to afford the isocyanate (695 mg) as a pale yellow oil.

The same procedure was used for the other 5-substituted 2-aminoaryl piperidine carboxamides.

### Example 8

This example illustrates synthesis of (5-bromophenyl)-(3,5-dimethyl-piperidin-1-yl)-methanone-2-ureas.

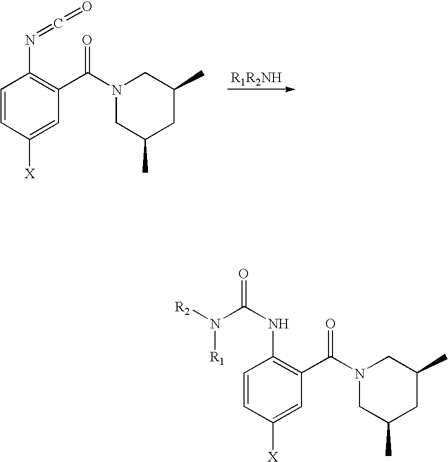

The isocyanate from example 7 was dissolved in about 1:1 mixture of dichloromethane/dimethylformamide at a concentration of about 0.1 mmol/ml. About 0.5 ml of this solution was added to the primary or secondary amine in an 8 ml screw cap vial. In the case of HCl or TFA salts, diisopropylethylamine (0.15 mmol) was added to each vial. The vials were capped and placed on a shaker at RT overnight. The solvent was then removed under reduced pressure and the reaction mixture dissolved in 1 ml dimethylformamide. The products were purified by HPLC-MS.

The following representative amines were used
glycine dimethylamide-TFA salt,
alanine dimethylamide-TFA salt,
d-proline dimethylamide TFA salt,
1-proline dimethylamide TFA salt,
sarcosine-dimethylamide TFA salt,
glycine morpholinamide-TFA salt,
alanine morpholinamide-TFA salt,
d-proline morpholinamide TFA salt,
1-proline morpholinamide TFA salt,
sarcosine-morpholinamide TFA salt,
(2,5-dimethyl-3-furyl)methylamine,
(1-methyl-1H-pyrrol-2-yl)methylamine,
(5-methyl-3-isoxazolyl)methylamine,
(1,5-dimethyl-1H-pyrazol-3-yl)methylamine,
3-thienylmethylamine,
(1-methyl-1H-imidazol-5-yl)methylamine,
N-methyl-N-(3-pyridylmethyl)amine,
2-fluorobenzylamine,
2-thiophenemethylamine,
5-methylfurfurylamine,
n-methylfurfurylamine,
2-fluoro-n-methylbenzylamine,
2-picolylamine,
3-picolylamine,
4-(aminomethyl)pyridine,
N-benzylmethylamine,
furfurylamine,
4-(dimethylamino)benzylamine, and
2-(aminomethyl)-5-methylpyrazine.

### Example 9

This example illustrates synthesis of N-(4-bromo-2-((3R,5S)-3,5-dimethylpiperidine-1-carbonyl)phenyl)pyrrolidine-2-carboxamide hydrochloride.

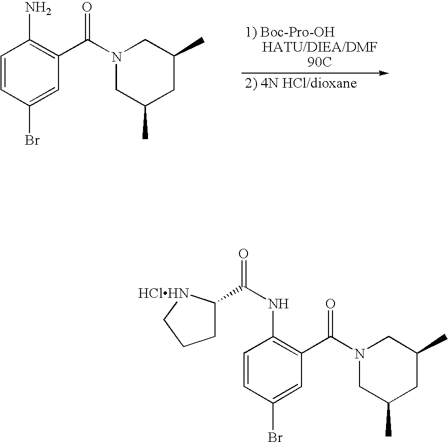

The (2-amino-5-bromophenyl)((3R,5S)-3,5-dimethylpiperidin-1-yl)methanone from example 6 or 7 (400 mg, 1.29 mmol, 1.2 equiv), Boc-Pro-OH (231 mg, 1.07 mmol, 1.0 equiv), HATU (1.29 mmol, 489 mg, 1.2 equiv), and DIEA (224 mg, 1.29 mmol, 1.2 equiv) were dissolved in DMF (5.0 mL) and heated at 90° C. overnight. The DMF was then removed and the residue purified by Isco CombiFlash Companion system (SiO₂, gradient 0 to 60% ethyl acetate in hexanes) to give 459 mg (84% yield) of the desired product. The Boc-protected compound (436 mg, 0.858 mmol, 1 equiv) was then dissolved in a solution of dioxane (2.5 mL) and 4N HCl (2.5 mL) and stirred at RT overnight. The resultant mixture was then lyophilized to afford the title compound according to LC-MS and ¹H NMR analysis.

The same procedure was used for BOC-D-proline, BOC-L-Proline, BOC-trans-4-hydroxyproline.

The same procedure was used with other (2-aminophenyl)((3R,5S)-3,5-dimethylpiperidin-1-yl)methanones bearing other 5-substituents, or other (3,5-mono or disubstituted) piperidines as in example 6 or 7.

### Example 10

This example illustrates acylation of N-(4-bromo-2-((3R,5S)-3,5-dimethylpiperidine-1-carbonyl)phenyl)pyrrolidine-2-carboxamide

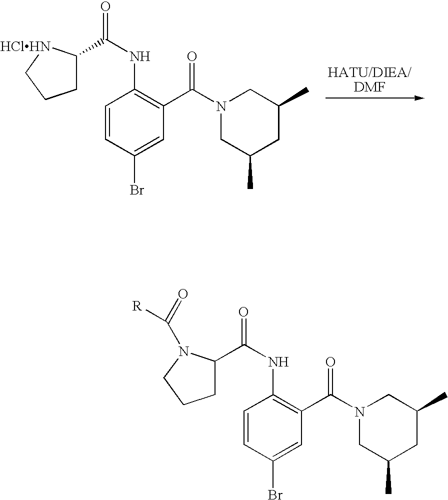

The pyrrolidine derivative from Example 9 was dissolved in about 1:1 mixture eof DCM/DMF. About 0.040 mmol (0.25 ml) of this solution was added to 0.075 mmol of the acyl chlorides in the table below in separate vials, followed by a solution of 0.100 mmol of DIEA in 0.25 ml of DCM/DMF. For the carboxylic acids, HATU (0.08 mmol) was added to each vial followed by the DIEA solution and the proline solution. The vials were capped and stirred overnight at RT. Scavenger resin (Argonaut PS-trisamine, 0.100 mmol) was added to each vial and the suspension stirred at RT for 3 hours, filtered and evaporated. The residue was redissolved in 1 ml of DMF and purified by reverse phase HPLC, and characterized by HPLC-MS.

The following acids or acid chlorides were purchased from a variety of suppliers and used as supplied:
Benzyl chloroformate,
Benzoyl chloride,
Propionyl chloride,
2,6-difluorobenzoyl chloride,
2-methoxybenzoyl chloride,
Methyl oxalyl chloride,
Cyclopropanecarbonyl chloride,
2-furoyl chloride,
Thiophene-2-carbonyl chloride,
Thiophene-2-acetyl chloride,
Pyrazine-2-carbonyl chloride,
1-methylpyrrole-2-carbonyl chloride,
Picolinic acid,
Nicotinic acid,
Isonicotinic acid,
Tetrahydro-2-furoic acid,
Imidazol-4-carboxylic acid,
Isoxazole-5-carboxylic acid,
Furazan-3-carboxylic acid,
5-methylisoxazole-3-carboxylic acid,
1-methyl-1H-imidazole-2-carboxylic acid,
(2-oxo-oxazolidin-3-yl)-acetic acid,
6-methyl-2-oxo-1,2-dihydro-pyrimidine-4-carboxylic acid,
Furan-2-carbonyl chloride,
Acetic Anhydride, and
4-Fluorobenzoyl chloride.

### Example 11

This example provides synthesis of acids derived from succinic anhydride.

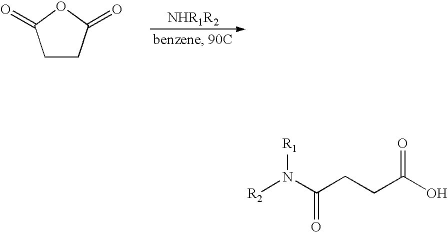

A solution of succinic anhydride (3.0 mmol, 1.2 equiv) and an amine compound (2.5 mmol, 1.0 equiv) in benzene (5 mL) was stirred at 90° C., overnight. The resulting mixture was concentrated under reduced pressure to afford the desired compounds in substantially quantitative yield according to LC-MS and ¹H NMR analysis.

The following amines were purchased from a variety of suppliers and used in these reactions as supplied:
1-acetyl piperazine,
N,N-diethylnipecotamide,
N-methylbenzyl amine,
N-methylfurfurylamine,
diethylamine,
Benzylamine,
4-methyl-piperazine,
(R)-1-phenyl-ethylamine,
(S)-1-phenyl-ethylamine,
2,6-dimethyl-morpholine,
morpholine,
piperidine,
thiomorpholine,
Dimethylamine,
N-methyl-pyridin-3-ylmethylamine, and
3-aminopyridine.

### Example 12

This example illustrates a general reaction procedure of carboxylic acids with 2-aminoaryl piperidine carboxamides.

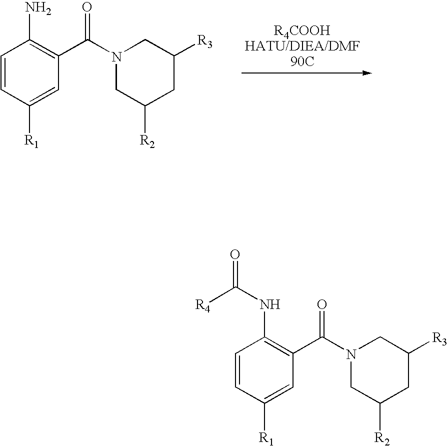

R₁=Br, Cl, OMe, and Me; R₂═R₃=Me (cis), CF₃; and R₂=Me, R₃=Et

In a parallel synthesis protocol, carboxylic acids from Example 11 (0.11 mmol, 1.5 equiv) were dispensed in reaction vials and dissolved in DMF (0.2 mL). A solution of HATU (0.14 mmol, 1.9 equiv) in DMF (0.2 mL) was then added to the solutions. A solution of the 2-aminoaryl piperidine carboxamide (0.075 mmol, 1 equiv) in DMF (0.2 mL) was then added, followed by diisopropyl ethylamine (DIEA) (0.15 mmol, 2.0 equiv). The reaction mixtures were agitated on a shaker at 90° C. for 16 h. The mixtures were concentrated and the residues dissolved in DMSO (1 mL) for automated analytical LC-MS analysis and preparative reversed-phase HPLC purification. The samples were subjected to analytical LCMS in order to confirm purity and product identity prior to submission to screening.

The following acids were purchased from a variety of vendors and used as supplied:
hippuric acid,
phenaceturic acid,
(1-(2-furylcarbonyl)-4-hydroxy-2-pyrrolidine carboxylic acid,
N,N-dimethylsuccinamic acid,
nicotinoyl-glycine,
N^{α}-acetyl-L-glutamine (Ac-Gln-OH),
N-acetylglycine,
N-acetyl-D,L-proline,
4-oxo-4-(3-pyridyl)butyric acid,
1-propionyl-pyrrolidine-2-carboxylic acid,
N-CbZ-Proline,
N-benzoyl-L-histidine,
N-(2-furoyl)glycine,
4-oxo-4-(2-thienyl)butyric acid,
N-acetyl-L-proline,
N-acetyl-Glutamine,
N-acetyl-D,L-alanine,
1-(4-Chlorobenzyl)-5-oxo-pyrrolidine-3-carboxylic acid,
1-Isobutyl-5-oxo-pyrrolidine-3-carboxylic acid,
1-Isopropyl-5-oxo-pyrrolidine-3-carboxylic acid,
3-Oxo-cyclopentanecarboxylic acid,
3-Oxo-indan-1-carboxylic acid,
4-Oxo-pentanoic acid,
(2-oxo-oxazolidin-3-yl)-acetic acid,
(3,4-dihydro-2H-benzo[b][1,4]dioxepin-7-yl)-4-oxo-butyramide,
1-(benzyloxycarbonyl)indoline-2-carboxylic acid,
4-oxo-4-pyridin-2-yl-butyric acid,
4-oxo-4-thiophen-2-yl-butyric acid, and
pyroglutamic acid.

### Example 13

This example illustrates a general reaction procedure of 2-aminoaryl piperidine carboxamides with succinic anhydride.

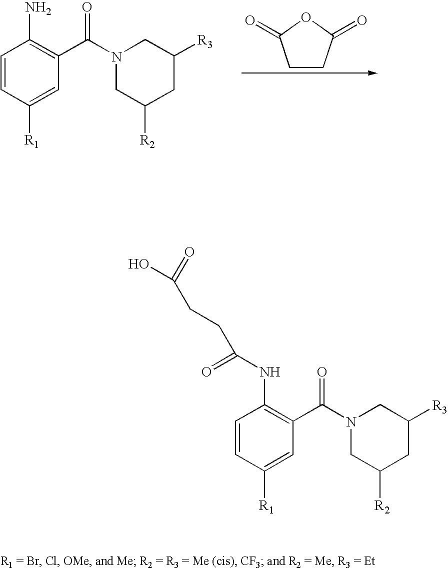

Succinic anhydride (0.11 mmol, 1.2 equiv) and the 2-aminoaryl piperidine carboxamide (0.092 mmol, 1.0 equiv) were dissolved in benzene (1 mL). The resulting solution was heated at 90° C. and agitated on a shaker overnight. The mixtures were concentrated (Genevac, Speed Vac) and the residues dissolved in DMSO (1 mL) for automated analytical LC-MS analysis and preparative reversed-phase HPLC purification. The samples were subjected to analytical LCMS in order to confirm purity and product identity prior to submission to screening.

### Example 14

This example illustrates a general procedure for the preparation of amino acid Amides.

To a solution of HATU (12 mmol) in 60 ml of DCM/DMF (1:1) was added DIEA (24 mmol). About 5 ml of this solution was added to each of the BOC protected amino acids (1 mmol per vial of either Glycine, DL-Alanine, D-Proline, L-Proline, Sarcosine). The solutions were stirred at RT for 15 minutes, then 1 ml of a 2 M solution of Dimethylamine in THF was added to vials 1-6 and 2 mmol and morpholine was added to vials 7-12. The reactions were stirred at RT for 3 hours. The solvents were removed under reduced pressure, and the residue dissolved in 10 ml of EtOAc, washed with satd. NaHCO₃, brine, and the organic layer dried with MgSO₄. The solvent was removed in vacuo, and the residues treated with 30% TFA/DCM for 1 hour at RT. The solvent was removed in vacuo and the products used in Example 8.

The following products were produced using this procedure:
GLYCINE dimethylamide-TFA,
DL-ALANINE dimethylamide-TFA,
D-PROLINE dimethylamide-TFA,
L-PROLINE dimethylamide-TFA,
SARCOSINE-dimethylamide-TFA,
Glycine morpholinamide-TFA,
DL-Alanine morpholinamide-TFA,
D-PROLINE morpholinamide-TFA,
L-PROLINE morpholinamide-TFA, and
Sarcosine-morpholinamide-TFA.

### Example 15

This example illustrates in vitro assay of HCV NS5B RNA Polymerase Activity

The enzymatic activity of HCV NS5B570n-BK is measured as incorporation of radiolabeled nucleotide monophosphates into acid insoluble RNA products. Unincorporated radiolabel substrate is removed by filtration and scintillant is added to the washed and dried filter plate containing radiolabeled RNA product. The light emitted by the scintillant is proportional to the amount of RNA product generated by NS5B570n-BK at the endpoint of the reaction.

The N-terminally histidine tagged HCV polymerase, derived from HCV BK strain, genotype 1b (NS5B570n-BK) contains a 21 amino acid deletion at the C-terminus relative to the full-length HCV polymerase and is purified from E. coli strain M15. The construct containing the coding sequence of HCV BK strain amino acid residues 2421-2999 (GenBank accession number M58335) downstream of a Taq promoter expression cassette was inserted into plasmid constructs. The plasmid constructs are transformed in E. coli and colonies are inoculated and grown overnight in 10 L of Terrific broth (Tartoff and Hobbs) supplemented with 100 μg/mL ampicillin at 37° C. Protein expression is induced by addition of 1 mM isopropyl-β-D-thiogalactopyranoside (IPTG), when optical densities reaches between 1.5 and 3.5 OD₆₀₀ and the culture is then incubated for 16- to 18 h at 22° C. NS5B570n-BK is purified to homogeneity using a three step protocol including subsequent column chromatography on Ni-NTA, SP-Sepharose HP and Superdex 75 resins.

Each 50 μl enzymatic reaction contains 8.4 μg/mL polyA:oligo U₁₆ (template:primer), 200 nM NS5B570n-BK enzyme, 2.1 μCi of tritiated UTP (Perkin Elmer catalog no. TRK-412; specific activity: 30 to 60 Ci/mmol; stock solution concentration from 7.5×10⁻⁵ M to 20.6×10⁻⁶ M), 1 μM ATP, CTP, and GTP, 40 mM Tris-HCl pH 8.0, 2 to 40 mM NaCl, 4 mM DTT (dithiothreitol), 4 mM MgCl₂, and 5111 of compound serial diluted in DMSO. Reaction mixtures are assembled in MADVNOB 96-well filter plates (Millipore Co.) and incubated for 2 h at 30° C. Reactions are stopped by addition of 10% (v/v) trichloroacetic acid and incubated for 40 min at 4° C. Reactions are filtered, washed with 6 reaction volumes of 10% (v/v) trichloroacetic acetic acid, 2 reaction volumes of 70% (v/v) ethanol, air dried, and 25 μl of scintillant (Microscint 20, Perkin-Elmer) is added to each reaction well.

The amount of light emitted from the scintillant is converted to counts per minute (CPM) on a Topcount® plate reader (Perkin-Elmer, Energy Range: Low, Efficiency Mode: Normal, Count Time: 1 min, Background Subtract: none, Cross talk reduction: Off).

Data is analyzed with GraphPad® Prism® and/or Microsoft® Excel®. The reaction in the absence of enzyme is used to determine the background signal, which is subtracted from the enzymatic reactions. Positive control reactions are performed in the absence of compound, from which the background corrected activity is set as 100% polymerase activity. All data is expressed as a percentage of the positive control. The compound concentration at which the enzyme-catalyzed rate of RNA synthesis is reduced by 50% (IC₅₀) is calculated by fitting equation (i) to the data, where “Y” corresponds to the relative enzyme activity (in %), “% Min” is the residual relative enzymatic activity at saturating compound concentration, “% Max” is the maximal relative enzymatic activity compared to positive control, X corresponds to the compound concentration, and “S” is the Hill coefficient (or slope).

$$Y = \% Min + \frac{( \% Max - \% Min )}{[ 1 + \frac{X}{{( {IC}_{50} )}^{S}} ]} \qquad ( i )$$

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

We claim:

1. A compound according to formula: 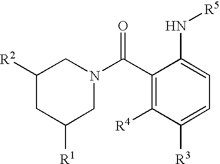
wherein
R¹ and R² are independently hydrogen, C₁₋₆ alkyl or C₁₋₆ haloalkyl;
R³ is hydrogen, halogen, C₁₋₆ alkyl or C₁₋₆ alkoxy;
R⁴ is hydrogen or halogen; and
R⁵ is a succinic acid moiety, a urea moiety, an amino acid moiety or an N—C₁₋₆ acyl amino acid moiety thereof.

2. The compound according to claim 1, wherein each of R¹ and R² is independently C₁₋₆ alkyl or C₁₋₆ haloalkyl.

3. The compound according to claim 2, wherein each of R¹ and R² is independently methyl, ethyl or trifluoromethyl.

4. The compound according to claim 1, wherein R¹ and R² are in a cis-configuration relative to each other.

5. The compound according to claim 1, wherein R³ is halogen, C₁₋₆ alkyl or C₁₋₆ alkoxy.

6. The compound according to claim 5, wherein R³ is halogen.

7. The compound according to claim 6, wherein R³ is Br, Cl, or I.

8. The compound according to claim 1, wherein R⁴ is hydrogen or F.

9. The compound according to claim 1, wherein R⁵ is selected from the group consisting of:
(i) C(═O)NR¹⁰R¹¹, wherein
R¹⁰ is hydrogen or C₁₋₆ alkyl; and
R¹¹ is:
(a) C₁₋₆ aralkyl, wherein the aryl group is optionally substituted with halogen, amino, C₁₋₆ monoalkylamino, or C₁₋₆ dialkylamino,
(b) C₁₋₆ heteroaralkyl, wherein the heteroaryl group is optionally substituted with alkyl, or
(c) CHR¹²CONR¹³R¹⁴, wherein
R¹² is selected from the group consisting of hydrogen or C₁₋₆ alkyl, and
R¹³ and R¹⁴ are independently hydrogen or C₁₋₆ alkyl,
or R¹³ and R¹⁴ taken together are (CH₂)₂O(CH₂)₂;
or R¹⁰ and R¹¹ taken together along with the nitrogen atom to which they are 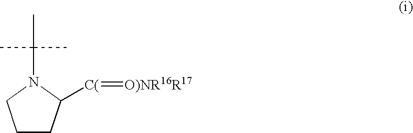
attached form a pyrrolidine moiety of formula (I) wherein each of R¹⁶ and R¹⁷ is independently hydrogen or C₁₋₆ alkyl, or, R¹⁶ and R¹⁷ taken together are —(CH₂)₂—O—(CH₂)₂—;
(ii) C(═O)CHR²⁰NR²¹R²² wherein
R²⁰ is hydrogen, C₁₋₆ alkyl, heteroalkyl, C₁₋₆ heteroaralkyl, (CH₂)ₙC(═O)NH₂ wherein n is 1 or 2, CH₂OH or 4-imidazol-4-yl-methyl;
R²¹ is hydrogen or C₁₋₆ alkyl;
R²² is —C(═O)R²³, wherein R²³ is C₁₋₆ alkyl, heteroaryl (which is optionally substituted with one or two substituents each of which is independently selected from the group consisting of C₁₋₆ alkyl, C₁₋₆ alkoxy, halogen, and morpholin-4-yl), C₁₋₆heteroaralkyl, amino, C₁₋₆ monoalkylamino, C₁₋₆ dialkylamino, aryl (which is optionally substituted with one or two substituents each of which is independently selected from the group consisting of C₁₋₆ alkyl and halogen), C₁₋₆ aralkyl, C₁₋₆ alkoxy, C₁₋₆ aralkoxy, heteroalkyl, tetrahydrofuran-2-yl, or heterocyclyl C₁₋₆ alkyl; or
R²⁰ and R²¹ together are (CH₂)₃, CH₂CH(OH)CH₂, or a moiety of the formula: 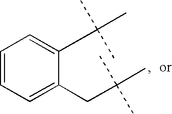
R²¹ and R²² together are C(═O)OCH₂CH₂ or CH₂CH₂OCH₂CH₂, or
R²⁰ and R²² together are C(═O)NR²⁴C(═O)CH₂, wherein R²⁴ is hydrogen or C₁₋₆ alkyl; and
(iii) C(═O)CHR³⁰CHR³¹COR³², wherein
R³⁰ and R³¹ are hydrogen; and
R³² is OR⁴⁰, NR³³R³⁴, C₁₋₁₆ alkyl, optionally substituted phenyl, 3,4-dihydro-2H-benzo[b][1,4]dioxepin-7-ylmethyl or heteroaryl;
wherein
each of R³³ and R⁴⁰ is independently hydrogen or alkyl,
R³⁴ is hydrogen, alkyl, optionally substituted aralkyl, heteroaralkyl, or heteroaryl; or
R³⁰ and R³¹ together are —(CH₂)ₙ—, wherein n is 1, 2, 3, or 4; or
R³⁰ and R³³ together are —CH₂—; or
R³⁰ and R³² together are optionally substituted 1,2-phenylene, —(CH₂)₂—; or
R³³ and R³⁴ together are —CH₂CHR⁴¹—X—CHR⁴²CH₂—, —(CH₂)₅—, —CH₂CHR³⁶—(CH₂)₃—;
X is NR³⁵, O or S;
R³⁵ is hydrogen, alkyl, or C₁₋₆ acyl;
each of R⁴¹ and R⁴² is independently hydrogen or alkyl;
R³⁶ is C(═O)NR³⁷R³⁸; and
each of R³⁷ and R³⁸ is independently hydrogen or alkyl.

10. The compound according to claim 9, wherein R⁵ is C(═O)NR¹⁰R¹¹, wherein R¹⁰ and R¹¹ are those defined in claim 9.

11. The compound according to claim 10, wherein R¹⁰ is hydrogen or methyl.

12. The compound according to claim 10, wherein R¹¹ is C₁₋₆ aralkyl, and wherein the aryl group is phenyl which is optionally substituted with one or two substituents each of which is independently selected from the group consisting of halogen, amino, monoalkylamino, and dialkylamino.

13. The compound according to claim 10, wherein R¹¹ is C₁₋₆ heteroaralkyl, and wherein the heteroaryl group is selected from the group consisting of:
pyridinyl,
1,5-dimethyl-1H-pyrazol-3-yl,
1-methyl-1H-pyrrol-3-yl,
5-methyl-isoxazol-3-yl, and
5-methyl-pyrazin-2-yl.

14. The compound according to claim 10, wherein R¹¹ is selected from the group consisting of benzyl, (1-methyl-1H-pyrrol-2-yl)methyl, (5-methyl-isoxazol-3-yl)methyl, (1,5-dimethyl-1H-pyrazol-3-yl)methyl, (pyridin-2-yl)methyl, (pyridin-3-yl)methyl, (pyridin-4-yl)methyl, 2-fluorophenylmethyl, 4-(N,N-dimethylamino)phenylmethyl, and a moiety of the formula: CHR¹²CONR¹³R¹⁴, wherein R¹² is hydrogen or methyl, and R¹³ and R¹⁴ are methyl or R¹³ and R¹⁴ together are —(CH₂)₂—O—(CH₂)₂—.

15. The compound according to claim 10, wherein R¹⁰ and R¹¹ taken together along with the nitrogen atom to which they are attached form a pyrrolidine moiety of the formula: 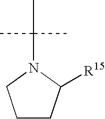
wherein
R¹⁵ is C(═O)NR¹⁶R¹⁷,
and wherein
each of R¹⁶ and R¹⁷ is independently hydrogen or C₁₋₆ alkyl,
or R¹⁶ and R¹⁷ taken together are —(CH₂)₂—O—(CH₂)₂—.

16. The compound according to claim 9, wherein R⁵ is C(═O)CHR²⁰NR²¹R²², wherein R²⁰, R²¹, and R²² are those defined in claim 9.

17. The compound according to claim 16, wherein R²⁰ is hydrogen, methyl, ethyl, hydroxymethyl, 1H-imidazol-4-ylmethyl, or a heteroalkyl of the formula: —(CH₂)ₙC(═O)NR²⁵R²⁶, wherein n is 1 or 2, and each of R²⁵ and R²⁶ is independently hydrogen or C₁₋₆ alkyl.

18. The compound according to claim 16, wherein R²¹ is hydrogen.

19. The compound according to claim 16, wherein R²² is —C(═O)R²³, wherein R²³ is methyl, pyridin-2-yl, 6-methylpyridin-2-yl, pyridin-3-yl, furan-2-yl, phenyl, benzyl, amino, iso-butyl, methoxy, 2-(morpholin-4-yl)pyridin-3-yl, benzyloxy, 4-fluorophenyl, tert-butoxy, ethyl, 2,6-difluorophenyl, thiophen-2-yl, thiophen-2-ylmethyl, pyrazin-2-yl, 1-methyl-1H-pyrrol-2-yl, pyridin-4-yl, tetrahydrofuran-2-yl, 3H-imidazol-4-yl, isoxazol-5-yl, furazan-3-yl, 5-methyl-isoxazol-3-yl, 1-methyl-1H-imidazol-2-yl, (2-oxo-oxazolidin-3-yl)methyl, 6-methyl-2-oxo-1,2-dihydropyrimidin-4-yl, 5-methylfuran-2-yl, 5-ethoxyfuran-2-yl, or 2-chloropyridin-3-yl.

20. The compound according to claim 9, wherein R⁵ is C(═O)CHR³⁰CHR³¹COR³², wherein R³⁰, R³¹ and R³² are those defined in claim 9.

21. The compound according to claim 1 of the formula: 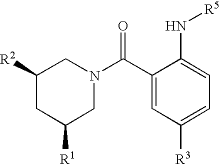
wherein
each of R¹ and R² is independently methyl, ethyl or trifluoromethyl;
R³ is halogen; and
R⁵ is as defined in claim 1.

22. A method for treating hepatitis C viral infection in a subject, said method comprising administering to a subject in need of such treatment a compound of claim 1.

23. A pharmaceutical composition comprising a compound according to claim 1 and at least one pharmaceutically acceptable carrier, diluent or excipient.
